(19) United States
(12) Patent Application Publication
(10) Pub. No.: US 2014/0128986 A1
(43) Pub. Date: May 8, 2014

(54) MONOBLOCK HEAD AND NECK UNIT FOR TOTAL HIP REPLACEMENT

(71) Applicant: iHip Surgical, LLC, Newport Beach, CA (US)

(72) Inventor: Anatol Podolsky, Corona Del Mar, CA (US)

(73) Assignee: iHip Surgical, LLC, Newport Beach, CA (US)

(21) Appl. No.: 14/073,796

(22) Filed: Nov. 6, 2013

Related U.S. Application Data
(63) Continuation-in-part of application No. 13/797,794, filed on Mar. 12, 2013.
(60) Provisional application No. 61/722,960, filed on Nov. 6, 2012.

(51) Int. Cl. A61F 2/36

(52) U.S. Cl. CPC A61F 2/3609
USPC 623/22.4

(57) ABSTRACT

Orthopedic hip replacement is made safer and more effective by enhancing interconnection of hip implant components, such as by improving an interference fit using one or more tapers, threads, and/or cooling of components prior to assembly. For example, a prosthetic femoral neck can include a thread and a Morse taper for lockable attachment to a prosthetic femoral head and/or intramedullary stem. A monoblock head and neck unit is described, having an integrated prosthetic femoral head and prosthetic neck, with structures for engaging the stem.

### RELATED APPLICATIONS

This application claims the benefit of priority from U.S. Provisional Application Ser. No. 61/722,960, filed on Nov. 6, 2012, and is a continuation-in-part of U.S. application Ser. No. 13/797,794, filed on Mar. 12, 2013, each of which is incorporated by reference in its entirety herein.

### FIELD

The embodiments herein relate generally to medical devices for use as hip arthroplasty implants.

### BACKGROUND

Failure of conventional joint implants can often be attributed to wear between components in the implant. For example, metal on metal fretting and wear can result in debris or corrosion being released from the implant. In certain instances, failure of conventional artificial hip implants can be attributed to wear between a modular femoral neck implant with a femoral head implant. In other instances, failure of conventional artificial hip implants can be attributed to wear between a modular femoral neck implant with a femoral stem or intramedullary rod implant. In some circumstances, metal on metal fretting and corrosion can lead to further damage. For example, in some conventional hip implants, fretting and/or crevice corrosion at the modular component junctions may occur. As loading is applied to the implant components from activities such as bearing weight, walking, and applying force at angles, relative micro-motion between the components can result in fretting, or wear of materials at pressure points at or near pivot points between the components. Conventional means of attaching a modular prosthetic neck can include tapping or hammering along the axis of a tapered connection, such as a Morse taper. Generally, conventional means of attaching components, such as the neck and stem or neck and head are difficult to align consistently and difficult to assemble using repeatable force. In some instances, conventional hip implants fail when the interface between the tapered surfaces are improperly aligned or seated, allowing rubbing, fretting, and wear resulting in the release of debris from the interface, which can result in increased blood serum metal levels, tissue inflammation, infection, pain, and/or necrosis. In some instances, these conventional designs can result in catastrophic failure.

### SUMMARY

Embodiments of the subject technology relate to medical methods and apparatus, and more particularly to a method and apparatus for attaching components in implants. In one embodiment, the components in an implant are attached in a manner to reduce fretting, debris, and/or material from wearing off the implant components. In one embodiment, a device includes a connection mechanism with a bore and cone interface. In one embodiment, a device includes a connection mechanism with a taper. In one embodiment, a device includes a connection mechanism with a thread. In one embodiment, a device includes a connection mechanism with a temperature differential. In various embodiments, any combination of features can used for a connection mechanism. In one embodiment, a device includes a connection mechanism between a prosthetic femoral neck implant to a prosthetic femoral head and/or prosthetic femoral stem implant in a total- or hemi-hip arthroplasty, and hip fracture fixation devices.

In accordance with some embodiments disclosed herein, various systems, components, and methods of use and surgery are provided to enhance the quality, reliability, and compatibility of implantation systems. These apparatuses and methods can be utilized for various types of implantation systems and methods of surgery, site and system preparation, and implantation. For example, embodiments of apparatuses disclosed herein for joint replacement may be used in joints of the human body. Embodiments of the methods disclosed herein can also be used for implanting medical devices in the body, such as prosthetic joints. These joints can include, but are not limited to the shoulder, the hip, the knee, etc. However, some embodiments can be provided in which the apparatuses and methods are used in other areas and with other structures. In some embodiments, implants are described in relation to a total hip arthroplasty. In some embodiments, implants are described in relation to a hemiarthroplasty, which includes a head replacement but no acetabular cup replacement.

In some embodiments, the subject technology offers a total or partial hip replacement system and a hip fracture treatment device in combination with truly minimally invasive surgical (MIS) technique. In some embodiments, both femoral neck and intertrochanteric hip fractures can be treated. In some embodiments, hemiarthroplasty can be performed with a femoral neck and intramedullary rod for intertrochanteric fracture fixation.

In one embodiment, an implant includes components that can be modularly attached to each other. In one embodiment, implant components can be attached with an taper interface. In various embodiments, the taper can be a Morse taper, or comprise a bore and cone and/or one or more sloped surfaces in the interface. In one embodiment, a modular prosthetic femoral neck has a head engaging portion that comprises a taper and a thread for engagement of a modular prosthetic femoral head to the prosthetic femoral neck implant. In one embodiment, the implant component interface can include a thread. In one embodiment, the implant component interface can include a locking thread. In one embodiment, the system includes a combination of propelling threads and locking Morse taper surfaces on an axis parallel to, or same as, the longitudinal axis of a modular prosthetic femoral neck. In one embodiment, the thread is configured to lock the femoral neck implant component in the femoral head implant with an interference fit between the thread and the at least one tapered surface. In one embodiment, the distal neck portion includes the head engaging end, and/or a head engaging portion. In one embodiment, the head engaging portion includes a Morse taper and a thread. In one embodiment, the thread redistributes the loading and point of potential micro-motion between the neck and head, creating one, two, three, four, or more pivot points and localizing potential fretting to an isolated, threaded location at the interface. In one embodiment, fretting and materials released by micro-motion is sealed, trapped, or contained within an interface. In one embodiment, fretting and materials are contained within an interface by a taper, such as a Morse taper surface. In one embodiment, the combination of a thread with the taper surfaces provides three, four, or more point bending that can prevent or reduce micro-motion and reduce fretting and corrosion of the modular connection. In one embodiment, the interface has a two point bending connection. In one embodiment, the interface has a three point bending connection. In one embodiment, the interface has a four point bending connection. In one embodiment, the interface includes a trunnion taper lock. In one embodiment, a combination of propelling threads and locking Morse taper surfaces on the same (or parallel) axis of the modular femoral neck will resolve inaccuracies of manual impaction of the head onto the neck at the trunnion interface; resulting in consistent reduction of fretting and corrosion.

In one embodiment, a modular prosthetic femoral neck has a stem engaging portion that comprises a taper and a thread for engagement of a modular prosthetic femoral stem to the prosthetic femoral neck implant. In one embodiment, the system includes a combination of propelling threads and locking Morse taper surfaces on an axis parallel to, or same as, the longitudinal axis of a modular prosthetic femoral neck. In one embodiment, the thread is configured to lock the femoral neck implant component in the femoral stem implant with an interference fit between the thread and the at least one tapered surface. In one embodiment, the distal neck portion includes the stem engaging end, and/or a stem engaging portion. In one embodiment, the stem engaging portion includes a Morse taper and a thread. In one embodiment, the thread redistributes the loading and point of potential micro-motion between the neck and stem, creating one, two, three, four, or more pivot points and localizing potential fretting to an isolated, threaded location at the interface. In one embodiment, fretting and materials released by micro-motion is sealed, trapped, or contained within an interface. In one embodiment, fretting and materials are contained within an interface by a taper, such as a Morse taper surface. In one embodiment, the combination of a thread with the taper surfaces provides three, four, or more point bending that can prevent or reduce micro-motion and reduce fretting and corrosion of the modular connection. In one embodiment, the interface has a three point bending connection. In one embodiment, the interface has a four point bending connection. In one embodiment, the interface includes a trunnion taper lock. In one embodiment, a combination of propelling threads and locking Morse taper surfaces on the same (or parallel) axis of the modular femoral neck will resolve inaccuracies of manual impaction of the stem onto the neck at the trunnion interface; resulting in consistent reduction of fretting and corrosion.

In one embodiment, a prosthetic femoral neck can be attached to both a prosthetic femoral head and a prosthetic femoral stem with both interfaces comprising at least a taper and a thread each.

In one embodiment, prosthetic femoral neck includes an interface for adjustable engagement with a driving tool. In one embodiment, the prosthetic femoral head implant is configured to fit rotatably within a prosthetic acetabular cup in the acetabulum. In one embodiment, prosthetic femoral head includes an interface for adjustable engagement with a driving tool.

In one embodiment, the method includes lowering the temperature of at least a portion of the femoral neck component, interconnecting the femoral neck component with a femoral head component and/or a femoral stem component, and permitting the temperature of the portion of the femoral neck component to rise such that an interference fit between the femoral neck component and the femoral head and/or stem component is increased. In one embodiment, the method includes lowering the temperature of at least a portion of a third component, interconnecting the portion of the third component with a portion of at least one of the femoral neck component and the femoral head or stem component in a second interference fit; and permitting the temperature of the portion of the third component to rise such that the interference fit between the third component and one of the femoral neck component and the femoral head or stem component is increased. In one embodiment, a method of interconnecting components of a prosthetic joint system includes lowering the temperature of at least a portion of a first component, interconnecting the first portion of the first component with a second component in an interference fit, and permitting the temperature of the portion of the first component to rise such that the interference fit between the first and second components is increased. In one embodiment, the method further includes lowering the temperature of at least a portion of a third component, interconnecting the portion of the third component with a portion of at least one of the first and second components in an interference fit, and permitting the temperature of the portion of the third component to rise such that the interference fit between the third component and one of the first and second components is increased. In one embodiment, the first component is a femoral neck component of a prosthetic hip system and the second component is a femoral head component. In one embodiment, the first component is a femoral neck component of a prosthetic hip system and the second component is a femoral stem component. In one embodiment, the first component and the second component are interconnected with at least one Morse taper.

In some embodiments, the subject technology offers an additional advantage with a prosthetic femoral neck that is attachable to a femoral stem. In one embodiment, a prosthetic femoral head is fixedly attached to the femoral neck. In one embodiment, a prosthetic femoral head is a monobody part of the femoral neck. In one embodiment, a prosthetic femoral head is modularly attachable to the femoral neck.

In some embodiments, the subject technology offers an additional advantage with a prosthetic femoral neck that extends from a first point external to the femur and through the femur to a second point where it joins the prosthetic femoral head. In some embodiments, a modular neck component that is inserted laterally through a bore in the stem provides advantages in reducing the amount of rotation, dislocation, and tissue damage that occurs in other techniques. In one embodiment, a prosthetic femoral neck having a head engagement end is configured to fixedly join the neck engagement portion of the prosthetic femoral head, the prosthetic femoral neck configured to be advanced from a position along a side of a patient's body, through a side of the femur opposite the acetabulum, and through a lateral bore of the intramedullary rod such that the head engagement end of the prosthetic femoral neck fixedly joins the neck engagement portion of the prosthetic femoral head while a portion of the prosthetic femoral neck occupies the lateral bore. In various embodiments, the prosthetic femoral neck can be rotated to actuate and/or connect to the prosthetic femoral head.

Some embodiments of the subject technology concern methods of performing a hip arthroplasty that can comprise some, or all of (1) surgically accessing an acetabulum, (2) preparing the acetabulum to receive a prosthetic acetabular cup (in embodiments with total hip arthroplasty), (3) seating the prosthetic acetabular cup in the prepared acetabulum, (4) fitting a prosthetic femoral head within the prosthetic acetabular cup (in embodiments with total hip arthroplasty), the prosthetic femoral head rotatable with respect to the prosthetic acetabular cup, (5) inserting a head-engaging end of a prosthetic femoral neck to engage the prosthetic femoral head, and (6) joining the head-engaging end of the prosthetic femoral neck to the prosthetic femoral head in any of the systems and methods disclosed herein. One embodiment further includes fixing the prosthetic femoral neck with respect to the prosthetic femoral head with a taper, such a Morse taper. One embodiment further includes fixing the prosthetic femoral neck with respect to the prosthetic femoral head with a thread. One embodiment further includes fixing the prosthetic femoral neck with respect to the prosthetic femoral head using a temperature differential.

Some embodiments of the subject technology concern methods of performing a hip arthroplasty that can comprise some, or all of (1) surgically accessing an acetabulum, (2) preparing the acetabulum to receive a prosthetic acetabular cup (in embodiments with total hip arthroplasty), (3) seating the prosthetic acetabular cup in the prepared acetabulum, (4) fitting a prosthetic femoral head within the prosthetic acetabular cup, the prosthetic femoral head rotatable with respect to the prosthetic acetabular cup, (5) inserting a stem-engaging end of a prosthetic femoral neck to engage the prosthetic femoral stem, and (6) joining the stem-engaging end of the prosthetic femoral neck to the prosthetic femoral stem in any of the systems and methods disclosed herein. One embodiment further includes fixing the prosthetic femoral neck with respect to the prosthetic femoral stem with a taper, such a Morse taper. One embodiment further includes fixing the prosthetic femoral neck with respect to the prosthetic femoral stem with a thread. One embodiment further includes fixing the prosthetic femoral neck with respect to the prosthetic femoral stem using a temperature differential.

Some methods may also derive advantages from an embodiment wherein an alignment tool comprises a first fixation keyway and the femoral neck comprises a second fixation keyway which removably interlocks with the first fixation keyway to facilitate removable fixation of the alignment tool to the neck and/or head. The method may derive additional advantage from an embodiment wherein the diameters of the prosthetic acetabular cup and the prosthetic femoral head both exceed 50 millimeters.

In some embodiments, a prosthetic joint system and methods of use can be provided that utilizes a unique interconnection between joint components to provide a stable coupling with superior strength and permanence. For example, in an embodiment of a hip prosthesis system, a prosthetic femoral neck can be coupled to a prosthetic femoral head and/or stem using one or more Morse tapers. In one embodiment, portions of the neck and head are threadably coupled to each other. Further, in some embodiments, one or more components of the system can be cooled and thereby shrunk prior to being interconnected such that the components are able to warm and expand upon implantation and interconnection. In some embodiments, the components of the system, such as the prosthetic femoral neck, can be frozen in liquid nitrogen prior to interconnection with the support sleeve. Accordingly, in some embodiments, the Morse tapers of the components can achieve a high degree of interference without requiring forcible insertion and trauma.

A monoblock apparatus for use as a hip arthroplasty implant for a patient that minimizes the likelihood of fretting and corrosion of the apparatus during use by the patient is provided. The apparatus is configured to be operably attached to a femoral stem and placed within the patient. The apparatus comprises a spherical head unit affixed to a femoral neck, the femoral neck comprising a Morse taper connection operably attached to a receptacle in the femoral stem in order to preserve modularity between the femoral neck and the femoral stem, wherein the spherical head unit, the femoral neck and the femoral stem may be adjusted in order to reproduce the patient's anatomy.

In certain embodiments of the subject technology, the monoblock apparatus comprises a spherical head unit affixed to a femoral neck. This allows the spherical head unit and the femoral neck to be integrated together as one piece. The head unit and femoral neck may be produced by net shape forging the head-neck components to a single unit, welding the components together, or by joining the components together using any other known technique in the field.

In surgery, a real implant comprising the components of the monoblock apparatus are assembled in situ. More specifically, a two-incision technique allows a surgeon to insert the femoral stem with the female Morse taper receptacle into the patient's femoral canal. The Morse taper connection of the monoblock apparatus is connected to the female Morse taper receptacle of the femoral stem through a separate anterior incision. In the alternative, the monoblock apparatus and femoral stem may be implanted into the patient through a single mini or regular incision.

The spherical head unit, the femoral neck, and the femoral stem come in different sizes to accommodate the length or offset required to reproduce the patient's anatomy and fit properly into the Acetabular cup inner diameter of the patient.

In some embodiments, a prosthetic hip system for hip arthroplasty comprises: a stem implant comprising an intramedullary rod and a distal bore extending into the stem implant from a distal bore end, the distal bore defining a tapered surface and a thread; a monoblock head and neck unit comprising a prosthetic femoral head, a tapered distal bore engaging portion configured to engage the tapered surface, and a threaded distal bore engaging portion configured to engage the thread.

In some embodiments, the intramedullary rod is configured to engage a femur of a patient, and the distal bore end is configured to face an acetabular region of the patient while the intramedullary rod engages the femur. In some embodiments, the monoblock head and neck unit further comprises a neck unit engagement portion configured to be engaged by a tool. In some embodiments, adjustment of the threaded distal bore engaging portion within the thread adjusts a degree of engagement between the tapered distal bore engaging portion and the tapered surface. In some embodiments, the tapered distal bore engaging portion is axially between the threaded distal bore engaging portion and the prosthetic femoral head. In some embodiments, the tapered surface is axially between the thread and the distal bore end. In some embodiments, an engagement between the tapered distal bore engaging portion and the tapered surface is configured to seal an interior region of the stem implant. In some embodiments, a first cross-sectional dimension at the distal bore end is greater than a second cross-sectional dimension at a location within the distal bore and proximal to the distal bore end.

In some embodiments, a method of implanting a prosthetic hip system for hip arthroplasty comprises: engaging a femur of a patient with an intramedullary rod of a stem implant; inserting a portion of a monoblock head and neck unit into a distal bore of the stem implant through a distal bore end, the monoblock head and neck unit comprising a prosthetic femoral head, a tapered distal bore engaging portion, and a threaded distal bore engaging portion; engaging the threaded distal bore engaging portion with a thread of the distal bore; and adjusting the threaded distal bore engaging portion relative to the thread, such that the tapered distal bore engaging portion engages a tapered surface of the distal bore.

In some embodiments, the method further comprises aligning the prosthetic femoral head with an acetabular region of the patient, such that the distal bore end faces the acetabular region. In some embodiments, a first cross-sectional dimension at the distal bore end is greater than a second cross-sectional dimension at a location within the distal bore and proximal to the distal bore end.

In some embodiments, a prosthetic hip system for hip arthroplasty comprises: a stem implant comprising an intramedullary rod, a distal bore extending into the stem implant from a distal bore end, the distal bore defining a distal tapered surface, and a proximal bore extending into the stem implant from a proximal bore end, the proximal bore defining a proximal tapered surface and intersecting the distal bore; a monoblock head and neck unit comprising a prosthetic femoral head, a tapered distal bore engaging portion configured to engage the distal tapered surface, and a first thread; and a proximal securing member comprising a tapered proximal bore engaging portion configured to engage the proximal tapered surface, and a second thread configured to engage the first thread.

In some embodiments, the intramedullary rod is configured to engage a femur of a patient, and the distal bore end is configured to face an acetabular region of the patient while the intramedullary rod engages the femur. In some embodiments, the monoblock head and neck unit further comprises a neck unit engagement portion configured to be engaged by a first tool, and wherein the proximal securing member comprises a securing member engagement portion configured to be engaged by a second tool. In some embodiments, adjustment of the first thread relative to the second thread adjusts a degree of engagement between the tapered distal bore engaging portion and the distal tapered surface and a degree of engagement between the tapered proximal bore engaging portion and the proximal tapered surface. In some embodiments, an engagement between the tapered distal bore engaging portion and the distal tapered surface and an engagement between the tapered proximal bore engaging portion and the proximal tapered surface is configured to seal an interior region of the stem implant. In some embodiments, a first cross-sectional dimension at the distal bore end is greater than a second cross-sectional dimension at a location within the distal bore and proximal to the distal bore end; and wherein a third cross-sectional dimension at the proximal bore end is greater than a fourth cross-sectional dimension at a location within the proximal bore and distal to the proximal bore end.

In some embodiments, a method of implanting a prosthetic hip system for hip arthroplasty comprises: engaging a femur of a patient with an intramedullary rod of a stem implant; inserting a portion of a monoblock head and neck unit into a distal bore of the stem implant through a distal bore end, the monoblock head and neck unit comprising a prosthetic femoral head, a tapered distal bore engaging portion, and a first thread; inserting a portion of a proximal securing member into a proximal bore of the stem implant through a proximal bore end, the proximal securing member comprising a tapered proximal bore engaging portion and a second thread; engaging the first thread with the second thread; and adjusting the first thread relative to the second thread, such that the tapered distal bore engaging portion engages a distal tapered surface of the distal bore and such that the tapered proximal bore engaging portion engages a proximal tapered surface of the proximal bore.

In some embodiments, the method further comprises aligning the prosthetic femoral head with an acetabular region of the patient, such that the distal bore end faces the acetabular region. In some embodiments, a first cross-sectional dimension at the distal bore end is greater than a second cross-sectional dimension at a location within the distal bore and proximal to the distal bore end; and wherein a third cross-sectional dimension at the proximal bore end is greater than a fourth cross-sectional dimension at a location within the proximal bore and distal to the proximal bore end. In some embodiments, the proximal securing member extends at least partially into the distal bore and the engaging the first thread with the second thread occurs in the distal bore.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates an exploded view of a prosthetic hip system with a prosthetic femoral head, a prosthetic femoral neck and an optional prosthetic acetabular cup in accordance with one embodiment of the subject technology.

FIG. 2 illustrates a temperature differential applied to a prosthetic femoral neck for attachment to a prosthetic femoral head in accordance with one embodiment of the subject technology.

FIG. 3 illustrates a prosthetic femoral head attachable to a prosthetic femoral neck with a thread and a tapered surface interface in accordance with one embodiment of the subject technology.

FIG. 4 illustrates a head tool for a prosthetic femoral head and a neck tool for a prosthetic femoral neck in accordance with one embodiment of the subject technology.

FIG. 5 illustrates an exploded view of a prosthetic hip system with a prosthetic femoral head, a prosthetic femoral neck and a prosthetic femoral stem in accordance with one embodiment of the subject technology.

FIG. 6 illustrates a temperature differential applied to a prosthetic femoral neck for attachment to a prosthetic femoral stem in accordance with one embodiment of the subject technology.

FIG. 7 illustrates a prosthetic femoral stem attachable to a prosthetic femoral neck with a thread and a tapered surface interface in accordance with one embodiment of the subject technology.

FIG. 8 illustrates a neck tool for a prosthetic femoral neck in accordance with one embodiment of the subject technology.

FIG. 9 illustrates a prosthetic femoral head attachable to a prosthetic femoral neck with a thread and a tapered surface interface, and a prosthetic femoral stem attachable to a prosthetic femoral neck with a thread and a tapered surface interface, in accordance with one embodiment of the subject technology.

FIG. 10A illustrates a top view of a prosthetic hip system with a prosthetic monoblock femoral head and a prosthetic femoral stem in accordance with one embodiment of the subject technology.

FIG. 10B illustrates a sectional view of a cross-section along line A-A of FIG. 10A, showing a prosthetic hip system with a prosthetic monoblock femoral head and a prosthetic femoral stem in accordance with one embodiment of the subject technology.

FIG. 10C illustrates a side view of a prosthetic monoblock femoral head in accordance with one embodiment of the subject technology.

FIG. 10D illustrates a sectional view of a cross-section along line B-B of FIG. 10C, showing a prosthetic monoblock femoral head in accordance with one embodiment of the subject technology.

FIG. 10E illustrates an exploded side view of a prosthetic hip system with a prosthetic monoblock femoral head and a prosthetic femoral stem in accordance with one embodiment of the subject technology.

FIG. 10F illustrates a side view of a prosthetic hip system with a prosthetic monoblock femoral head and a prosthetic femoral stem in accordance with one embodiment of the subject technology.

FIG. 11A illustrates a top view of a prosthetic hip system with a prosthetic monoblock femoral head, a proximal securing member, and a prosthetic femoral stem in accordance with one embodiment of the subject technology.

FIG. 11B illustrates a sectional view of a cross-section along line C-C of FIG. 11A, showing a prosthetic hip system with a prosthetic monoblock femoral head, a proximal securing member, and a prosthetic femoral stem in accordance with one embodiment of the subject technology.

FIG. 11C illustrates a side view of a prosthetic hip system with a prosthetic monoblock femoral head, a proximal securing member, and a prosthetic femoral stem in accordance with one embodiment of the subject technology.

FIG. 11D illustrates an exploded side view of a prosthetic hip system with a prosthetic monoblock femoral head and a prosthetic femoral stem in accordance with one embodiment of the subject technology.

FIG. 11E illustrates an exploded perspective view of a prosthetic hip system with a prosthetic monoblock femoral head and a prosthetic femoral stem in accordance with one embodiment of the subject technology.

FIG. 11F illustrates a side view of a prosthetic hip system with a prosthetic monoblock femoral head and a prosthetic femoral stem in accordance with one embodiment of the subject technology.

FIG. 11G illustrates a perspective view of a prosthetic hip system with a prosthetic monoblock femoral head and a prosthetic femoral stem in accordance with one embodiment of the subject technology.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

While the present description sets forth specific details of various embodiments, it will be appreciated that the description is illustrative only and should not be construed in any way as limiting. Additionally, it is contemplated that although particular embodiments of the subject technology may be disclosed or shown in the context of hip surgeries, such as total hip arthroplasty or hemiarthroplasty, such embodiments can be used in other surgical techniques and devices. Furthermore, various applications of such embodiments and modifications thereto, which may occur to those who are skilled in the art, are also encompassed by the general concepts described herein.

Embodiments of the methods, systems, components, and devices disclosed herein can be used for various joints of the body, such as the shoulder, hip, and the like. As discussed in the above-noted publications, joint replacements for the hip are common and have several factors that can be considered when designing a hip prosthetic system and methods of implantation. In the present disclosure, reference is made to a prosthetic hip joint and system. However, the systems and methods disclosed herein can be used for various joints in the body. Thus, the present disclosure should be construed as applicable to methods, systems, components, and devices for any of the various joints of the body, such as the shoulder, hip, and the like.

There is a need for an improved method and device for attaching components in implants. There is a need for an improved method and device for connections in implants that align the components and are able to apply controllable, reproducible force to engage the component connections. There is also a need for an improved method and device for attaching hip implants that use tapered connections, such as a femoral neck and/or stem and/or head, to other components in modular hip replacements.

In various embodiments, implants can include attachable components with interfaces. In one embodiment, a taper is included in an interface between implant components. In one embodiment, a taper comprises tapered surfaces, such as with a bore and a cone surface that complement each other. In one embodiment, the taper is a Morse taper. In one embodiment, a thread is included in an interface between implant components. In one embodiment, the thread is a locking thread. In one embodiment, a locking thread is configured to improve reliability of an interface connection under vibration. In one embodiment, a thread can lock the interface between tapered surfaces between implant components. In one embodiment, a thread can control the relative position and/or rotation of the bore and the cone to engage a taper via relative rotation. In one embodiment, a thread provides a controllable interface between tapered surfaces between implant components. In one embodiment, a thread can provide proper alignment between the bore and the cone to engage a tapered interface. In one embodiment, a thread can provide the ability to control a taper engagement force.

It will be appreciated that various surgical approaches may be used to access the femoral neck and acetabulum regions, and the subject technology is not limited by any particular surgical approach. Nor is the subject technology limited by any particular material for the prosthetic femoral head, prosthetic femoral neck, prosthetic femoral stem and/or an optional acetabular cup. Any of the components may be made from cobalt chromium, titanium, tantalum, surgical grade stainless steel, ceramic, alumina ceramic or other materials and/or alloys of suitable strength and acceptance properties.

In accordance with various embodiments, a prosthetic hip system 10 is provided for a minimally invasive, hip arthroplasty procedure. FIG. 1 illustrates an embodiment of a prosthetic hip system 10 with a prosthetic femoral head 100 positionable in an optional prosthetic acetabular cup 50. In one embodiment, a hemi-hip arthroplasty involves the attachment of a prosthetic femoral head 100 to a prosthetic femoral neck 200 implant. In one embodiment, a total hip arthroplasty further includes a prosthetic acetabular cup 50, which is seated in the acetabulum of the pelvis and is configured to allow rotational motion by the prosthetic femoral head 100. Although some figures may show a prosthetic acetabular cup 50, some embodiments of the subject technology do not need to include a prosthetic acetabular cup 50.

In one embodiment, a prosthetic femoral head 100 is fit into a prosthetic acetabular cup 50. In one embodiment, the prosthetic femoral head 100 at a cup-engaging end 110 comprises a partial sphere having a curvature machined to precisely fit the inner surface of the prosthetic acetabular cup 50. The partial sphere of the prosthetic femoral head 100 may extend, in various embodiments from approximately 160 degrees to approximately 340 degrees, and thus may comprise any range from somewhat less than a hemisphere to nearly a full sphere. In one embodiment, the partial sphere of the prosthetic femoral head 100 is placed against the exposed rim of the hemispherical inner surface of the prosthetic acetabular cup 50. As will be appreciated, one or more light taps using a firm rubber-headed impacting tool may then seat the prosthetic femoral head properly into the prosthetic acetabular cup 50.

In one embodiment, the prosthetic femoral head 100 at a neck engaging end 120 includes structural means to receive and engage a prosthetic femoral neck 200. In a preferred embodiment, neck engagement may be achieved by a very slightly and narrowingly tapered cylindrical neck bore 122 machined approximately 2 cm into the prosthetic femoral head from the neck engaging end 120 inward toward the center of the prosthetic femoral head 100, such that a head-engaging end 220 of a prosthetic femoral neck 200 comprising roughly 2 cm of cylindrical shaft having a Morse taper matched to that of the neck bore 122 may be driven by impact into the neck bore 122, resulting in a fit sufficiently permanent to operatively support load-bearing movement about the prosthetic hip without slippage. In one embodiment, a neck bore 122 may extend more than or less than 2 cm into the prosthetic femoral head 100, and the head-engaging end 220 of the prosthetic femoral neck 200 will be of a roughly corresponding length of more than or less than 2 cm. Also, the diameter of the neck bore 122 will be approximately 11-13 mm (and will very gradually decrease as the bore extends into the prosthetic femoral head to accommodate the taper), although it will be appreciated that smaller or larger diameters may be used, and it will also be appreciated that the shaft diameter of the head-engaging end of the prosthetic femoral neck 200 will be of a diameter matching that of the neck bore 122.

In another embodiment (not shown), a different attachment technique may be used to join the prosthetic femoral head 100 to a prosthetic femoral neck 200. For example, the prosthetic femoral head 100, rather than include a neck bore 122, may include a neck shaft. The neck shaft may extend approximately 2 cm outward from the neck-engaging end 120 of the prosthetic femoral head 100. The neck shaft may be approximately 11-13 mm in diameter (though smaller or larger diameters could be used), with the diameter slightly decreasing along the neck shaft in the direction away from the center of the prosthetic femoral head, to form a Morse taper. It will be appreciated that a prosthetic femoral neck in approximately the form of a cylindrical shaft, may be machined to include a bore in one end having a receiving Morse taper of proper dimension to engage the neck shaft. It will be appreciated that still further methods and structures exist that could be adapted to the prosthetic femoral head and prosthetic femoral neck to facilitate the joining of these two prostheses.

In various embodiments, the neck bore 122 is any shaped interface. In one embodiment, the neck bore 122 is round. In one embodiment, the neck bore 122 is oval. The neck bore 122 is configured to receive the neck implant 200. The neck bore 122 can comprise one or more registration structures to rotationally secure the neck implant 200. The registration structures can comprise one or more protrusions and/or recesses extending along an outer surface of the neck implant 200 and/or the neck bore 122. In one embodiment, the neck bore 122 includes one or more threads or threaded portions. In one embodiment, the neck bore 122 includes one, two, or more tapered surfaces 124. In one embodiment, the tapered surface 124 is a Morse taper. In one embodiment, the distal bore end 5134 includes one, two, or more tapered surfaces 124. In one embodiment, the tapered surface 124 is a Morse taper. In various embodiments, the taper 124 is configured to seal the interface between system parts to prevent the escape of debris or flaking from components that may rub against each other in use. In various embodiments, the taper 124 is configured to provide an adjustable interface to account for differences in tolerances in dimensions between parts or components.

In accordance with various embodiments, a prosthetic hip system 10 is provided for a minimally invasive, hip arthroplasty procedure. FIG. 5 illustrates an embodiment of a prosthetic hip system 10 with a prosthetic femoral head 100, a prosthetic femoral neck 200 and a prosthetic femoral stem 300. In various embodiments, any of the prosthetic femoral neck 200 and either the prosthetic femoral head 100 or prosthetic femoral stem 300 can be permanently attached or constructed from a monolithic material. In some embodiments, only a prosthetic femoral head 100 can be attached to the prosthetic hip system 10, such as through a threaded interface in which the prosthetic femoral head 100 is rotated about a thread. In some embodiments, only a prosthetic femoral neck 200 can be attached to the prosthetic femoral stem 300, such as through a threaded interface in which the prosthetic femoral neck 200 is rotated about a thread.

In one embodiment, the prosthetic femoral stem 300 at a neck engaging end 320 includes structural means to receive and engage a prosthetic femoral neck 200. In a preferred embodiment, neck engagement may be achieved by a very slightly and narrowingly tapered cylindrical neck bore 322 machined approximately 2 cm into the prosthetic femoral head from the neck engaging end 320 inward toward the center of the prosthetic femoral stem 300, such that a stem-engaging end 225 of a prosthetic femoral neck 200 comprising roughly 2 cm of cylindrical shaft having a Morse taper matched to that of the neck bore 322 may be driven by impact into the neck bore 322, resulting in a fit sufficiently permanent to operatively support load-bearing movement about the prosthetic hip without slippage. In one embodiment, a neck bore 322 may extend more than or less than 2 cm into the prosthetic femoral stem 300, and the stem-engaging end 225 of the prosthetic femoral neck 200 will be of a roughly corresponding length of more than or less than 2 cm. Also, the diameter of the neck bore 322 will be approximately 11-13 mm (and will very gradually decrease as the bore extends into the prosthetic femoral stem to accommodate the taper), although it will be appreciated that smaller or larger diameters may be used, and it will also be appreciated that the shaft diameter of the stem-engaging end 225 of the prosthetic femoral neck 200 will be of a diameter matching that of the neck bore 322.

In some embodiments, a tapered surface 124, 224, 324 can be a Morse taper. In various embodiments, the taper 124, 224, 324 can be in the range of 0-10 degrees, 1-9 degrees, 2-8 degrees, 4-7 degrees, 5-6 degrees. In various embodiments, one, two or more tapers 124, 224, 324 can extend along between about 0.1-3, 0.5-2, 1-1.5 cm and/or less than or equal to about 3 cm of the distal neck portion 210 and/or a proximal neck portion of the femoral neck implant component 200. In various embodiments, one, two or more tapers 124, 224, 324 can extend along about 2 cm of a component. In various embodiments, the diameter of the bore can be between at least about 10 mm and/or less than or equal to about 17 mm. In some embodiments, the diameter of the bore can be between at least about 11 mm and/or less than or equal to about 15 mm. Further, the diameter of the distal section of the bore can be between at least about 10 mm and/or less than or equal to about 17 mm.

In one embodiment, a prosthetic femoral neck 200 may be a straight shaft, which may be slightly tapered on one end to fixedly join a prosthetic femoral head 100 by insertion into a neck bore 122 (see FIG. 1 and related description), and/or which may be slightly tapered on one end to fixedly join a prosthetic femoral stem 300 by insertion into a neck bore 322 (see FIGS. 5, 6 and related description). In one embodiment, a prosthetic femoral neck 200 may have a circular cross section. It will be appreciated that the cross-sectional shape may differ, and other embodiments are specifically contemplated such as, for example, oval, square, rectangular, triangular, irregular or other cross-sectional shapes may be used, where the shape of the neck bore 122 in the prosthetic femoral head 100 and/or the neck bore 322 in the prosthetic femoral stem 300 is configured to correspondingly receive a prosthetic femoral neck 200 having such cross-sectional shape. While a circular cross-section of a head-engaging end 220 of a prosthetic femoral neck may be used with the remainder of the prosthetic femoral neck 200 and/or a stem-engaging end 225 having a different cross-sectional shape, in another embodiment the neck-receiving bore 122 in the prosthetic femoral head 100 may be configured to receive a head-engaging end 220 of a prosthetic femoral neck 200 having a cross-sectional shape other than circular. In one embodiment, the neck-receiving bore 322 in the prosthetic femoral stem 300 may be configured to receive a stem-engaging end 225 of a prosthetic femoral neck 200 having a cross-sectional shape other than circular. In another embodiment, a prosthetic femoral neck 200 may be curved and/or may include fixation grooves. It will be appreciated that the prosthetic femoral neck 200 may be used to facilitate advantageous angling of the femoral head and/or femoral stem and also may be used for right or left hip joint repair simply by flipping it upside down.

In various embodiments of a prosthetic hip system 10, a prosthetic femoral neck 200 is attachable to another component, such as a prosthetic femoral head 100 and/or a prosthetic femoral stem 300, wherein the components are attached with one, two, or more interfaces, threads, locks, pins, locking screws, top locking screws, seals, adhesives, glues, cement, temperature differentials, cold welding, interference fits, tapers, Morse tapers, impacting, tapping, hammering, and/or other attachment mechanisms. In various embodiments, the prosthetic hip system 10 may have one, two, three, or more components, parts, portions, features, or sub-components that are attachable that include one, two, or more interfaces, threads, locks, pins, locking screws, top locking screws, seals, adhesives, glues, cement, temperature differentials, cold welding, interference fits, tapers, Morse tapers, impacting, tapping, hammering, and/or other attachment mechanisms. According to some embodiments, methods and systems for providing stable and secure interconnection of components are provided. Some embodiments can utilize structural interconnections that create press or interference fits between interlocking components. Some embodiments can utilize rotational or translational couplings that involve the use of torque or other force to engage the components. In various embodiments, any components can be joined or attached in any way—for example, the neck implant 200 connectable to a head implant 100 and/or a stem implant 300, or any components, parts, portions, features, or sub-components thereof.

Further, some embodiments can utilize joining techniques that can enhance the interconnection of the components, such as by the use of temperature differential through heating or cooling the components to enhance a press, taper and/or interference fit. In various embodiments, components can be manufactured from the same or different materials in order to achieve desired characteristics and temperature-dimensional responsiveness. In some embodiments, at least a portion of one or more of interconnecting components can be cooled, such as by a nitrogen bath, to cause interconnecting aspects of the component to be reduced in size or dimension prior to being coupled with the other component. For example, once cooled, the interconnection aspects can be coupled to achieve a maximum press or interference fit in a cooling stage. Thereafter, as the component warms and expands, the engagement provided by the press or interference fits can be enhanced as dimensions of the interconnecting aspects of the components increase, thereby enhancing the interference and contact between the interconnecting aspects of the components.

As shown in FIGS. 2 and 6, in some embodiments, a temperature differential 400 can be applied to one or more components to expand or shrink a component material or part, such that upon equalization of temperature an interference fit, cold-weld, or other attachment holds or supplements the connection between the components. A living human body has a body temperature of roughly 37 degrees Celsius. Various compositions or materials are available in the operating room to cool components. For example, a ratio of 1:2.5 of CaCl2.6H2O/ice is roughly −10 degrees Celsius, a ratio of 1:3 of NaCl/ice is roughly −20 degrees Celsius, carbon tetrachloride/CO2 is roughly −23 degrees Celsius, acetonitrile/CO2 is roughly −42 degrees Celsius, a ratio of 1:0.8 CaCl2.6H2O/ice is roughly −40 degrees Celsius, Acetone/CO2 is roughly −78 degrees Celsius, Methanol/N2 is roughly −98 degrees Celsius, and liquid nitrogen (Liquid N2) is roughly −196 degrees Celsius. In one embodiment, a freezer or refrigerating unit is used to cool a component.

In one embodiment, a temperature differential 400 includes cooling a component of the prosthetic hip system 10 and/or tooling associated with the prosthetic hip system 10. Once the cooled component is implanted in vivo, the body temperature of the patient warms the cooled component, resulting in some material expansion to improve a connection between components. In various embodiments, cooling through a temperature differential 400 can benefits that include less-traumatic hammering, less damage, automatically locking features, improved connection through a cold weld, reduction in component material flaking or debris, reduction in dispersal of flaking or debris, minimal damage to tissue, materials such as metals tend to equalize in temperature through thermal conduction before tissue is damaged. In one embodiment, cooling of one or more parts or components through a temperature differential 400 can cause condensation or the formation of moisture from the surrounding air, which can act as a lubricant to aid the insertion or implantation process.

In one embodiment, as shown in FIG. 2, the prosthetic femoral neck 200 implant is cooled and inserted in to a prosthetic femoral head 100. In one embodiment, as shown in FIG. 6, the prosthetic femoral neck 200 implant is cooled and inserted in to a prosthetic femoral stem 300. When the prosthetic femoral neck 200 implant warms, it expands and further locks the prosthetic femoral head 100 and/or stem 300 to the prosthetic femoral neck 200, such as in one embodiment, by engaging a taper. In one embodiment, the femoral neck implant 200 can be cooled prior to installation into the bore of the head implant 100 and/or stem implant 300 in order to create material shrinkage of the neck implant 200. In one embodiment, the size of the neck implant 200 can be reduced such that upon installation, the neck implant 200 can heat up and expand to create an interference fit with the bore of the neck engaging end 120 of the prosthetic femoral head 100 by virtue of the expanding size of the neck within the bore. In one embodiment, the size of the neck implant 200 can be reduced such that upon installation, the neck implant 200 can heat up and expand to create an interference fit with the bore of the neck engaging end 320 of the prosthetic femoral stem 300 by virtue of the expanding size of the neck within the bore. In various embodiments, additional parts or sub-components in the prosthetic hip system 10 can have temperature differentials 400 applied to improve the connection between parts or sub-components. Combinations of cooling with one, two or more tapers, threads, or other features are contemplated. Some embodiments can provide advantages that are superior to some traditional interfaces that may be driven together by impact or force in order to create in a fit sufficiently permanent to operatively support load-bearing movement about the prosthetic hip without slippage. Although such interface joining techniques can provide a tight fit, such structures and methods of use involve a high degree of force and can be undesirable for providing a careful, yet secure installation procedure. In contrast, embodiments disclosed herein provide exceptional engagement and fit. Further, some embodiments provide superior engagement using a unique cooling process to achieve maximum interference between mated surfaces and features of the components of the system.

As shown in FIG. 3, in one embodiment, a prosthetic hip system 10 includes a neck implant 200 with a neck thread 250 that is connectable to a head implant 100 with a head thread 150. In one embodiment, the threads 150, 250 provide a tightenable, locking interface. In one embodiment, the threads 150, 250 are reversible for disassembly. In one embodiment, the threads 150, 250 operate in conjunction with a tapered surface to attach a neck implant 200 to a head implant 100. In one embodiment, the tapered surfaces 124, 224 are complementary Morse tapers. In one embodiment, a temperature differential 400 is applied to the threaded prosthetic hip system 10.

In one embodiment, the threaded prosthetic hip system 10 is assembled by inserting the prosthetic femoral neck 200 in to the prosthetic femoral head 100 and rotating the neck 200 and head 100 with respect to each other to engage the complementary threads 150, 250. As the threads 150, 250 bring the head 100 and neck 200 together, complementary tapered surfaces 124, 224 can engage each other. With the threaded interface, hammering is not necessary. With the threaded interface, a precise, repeatable attachment can performed with higher precision. In one embodiment, a tool can be configured to deliver a precise or maximum torque to tighten the threads.

In one embodiment, the threads 250, 150 are positioned at a distal end or near the distal end of the a prosthetic femoral neck 200 head engaging end 220 and the neck bore 122 in the prosthetic femoral head 100. One advantage of positioning threads at the distal end of the interface is that fretting or debris resulting from micro-motion of the interface localized to the threads will be trapped or contained within the distal end of the interface. In other embodiments, the threads 250, 150 can be positioned at any point, proximal, medial, distal, or otherwise along the prosthetic femoral neck 200 head engaging end 220 and the neck bore 122 in the prosthetic femoral head 100.

In one embodiment, a modular, threaded prosthetic hip system 10 includes a prosthetic femoral neck 2000 with a head engaging portion 220 that comprises a taper 224 and a thread 250 for attachable engagement to a modular prosthetic femoral head 100 to the prosthetic femoral neck 200 implant. In one embodiment, the system 10 includes a combination of propelling threads 150, 250 and locking Morse taper surfaces 124, 224 on an axis parallel to, or same as, the longitudinal axis of a modular prosthetic femoral neck 200. In one embodiment, the threads 150, 250 are configured to lock the femoral neck implant 200 component to the femoral head implant 100 with an interference fit between the threads 150, 250 and the at least one tapered surface 124, 224. In one embodiment, the distal neck portion includes the head engaging end, and/or a head engaging portion. In one embodiment, the head engaging portion includes a Morse taper and a thread. In one embodiment, the thread 150, 250 redistributes the loading and point of potential micro-motion between the neck 200 and head 100, creating one, two, three, four, or more pivot points and localizing potential fretting to an isolated, threaded location at the interface. In one embodiment, fretting and materials released by micro-motion are sealed, trapped, or contained within the interface. In one embodiment, fretting and materials are contained within an interface by a taper, such as a Morse taper surface. In one embodiment, the combination of a thread with the taper surfaces provides one, two, three, four, or more point bending that can prevent or reduce micro-motion and reduce fretting and corrosion of the modular connection. In one embodiment, the interface has a two point bending connection. In one embodiment, the interface has a three point bending connection. In one embodiment, the interface has a four point bending connection. In one embodiment, the interface includes a trunnion taper lock. In one embodiment, a combination of propelling threads and locking Morse taper surfaces on the same (or parallel) axis of the modular femoral neck will resolve inaccuracies of manual impaction of the head onto the neck at the trunnion interface; resulting in consistent reduction of fretting and corrosion.

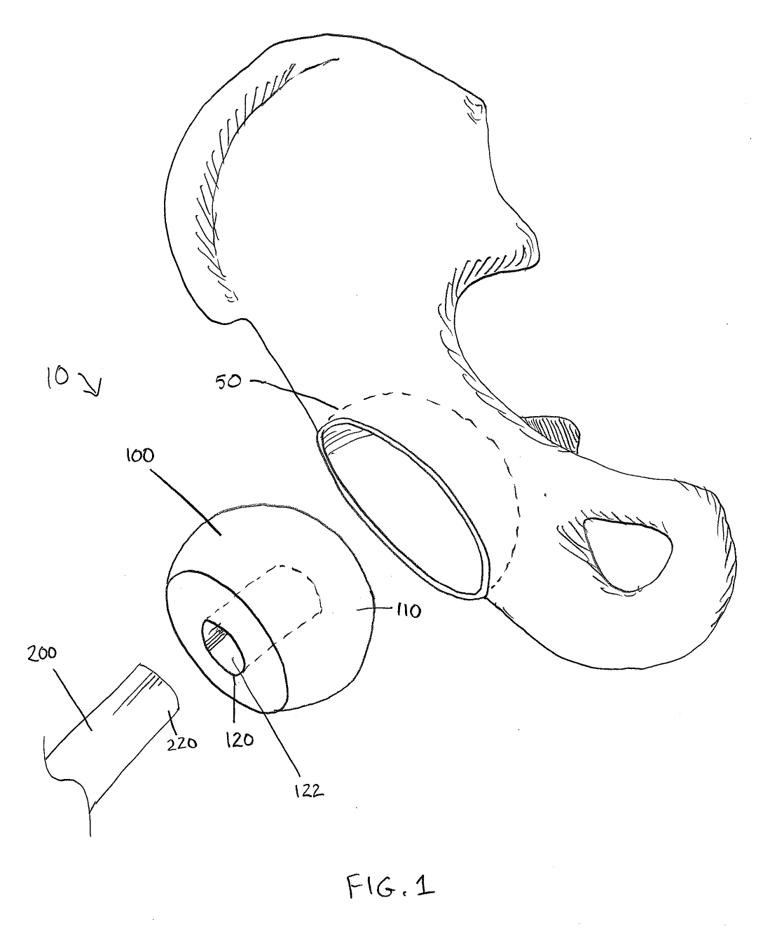

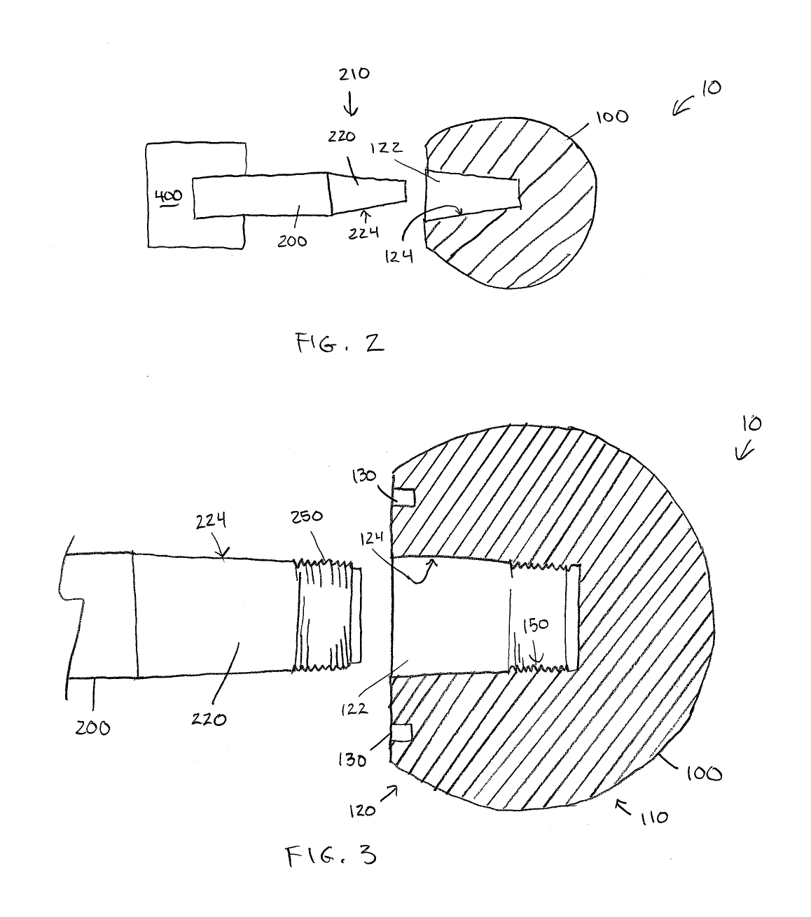

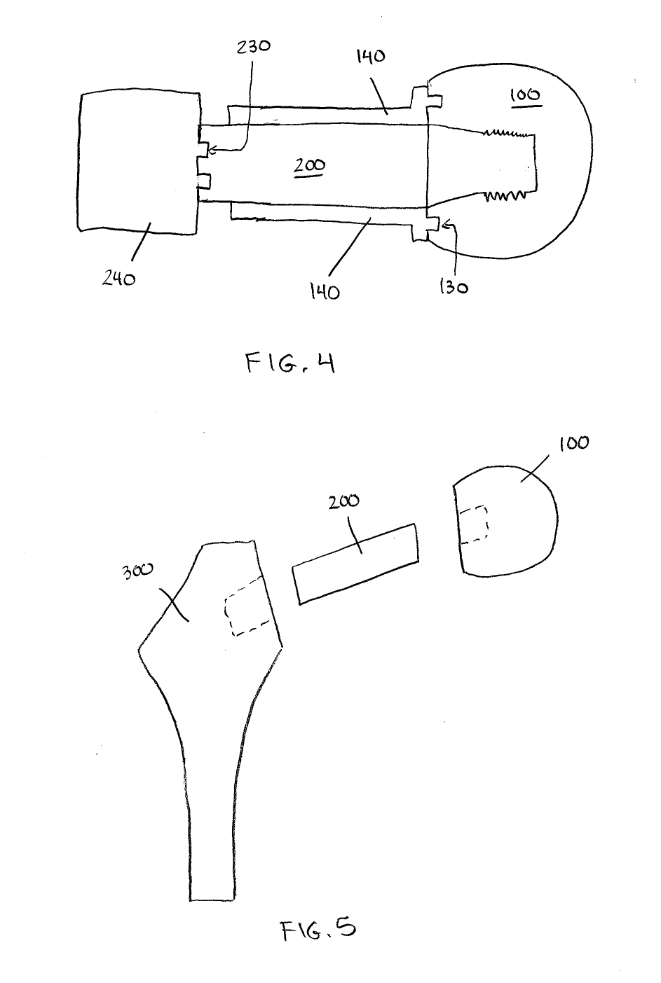

As shown in FIG. 4, in one embodiment, the prosthetic femoral neck 200 includes a neck tool engaging portion 230 configured for a neck tool 240 for implantation, actuation, assembly, rotation, threading, and/or removing the prosthetic femoral neck 200. In various embodiments, the neck tool engaging portion 230 is a slot, keyed interface, hexagonal, or other interface for rotating the prosthetic femoral neck 200 to engage the neck thread 250 with the head thread 150. In one embodiment, the neck tool engaging portion 230 is on a proximal end of the prosthetic femoral neck 200, and the neck tool engaging portion 230 includes features for rotatable engagement. In various embodiments, the neck tool 230 can apply 0-5000, 0-4000, 0-500, 0-2000, 0-1000, 0-100, 10-80, 20-70, 30-60, 33, 45, and/or 55 ft-lb of torque to the neck thread 250.

In one embodiment, a head tool 140 includes one or more pins, keys, or other interface to hold the prosthetic femoral head 100 in position while a threaded prosthetic femoral neck 200 is threaded to the head 100. In one embodiment, no neck tool 240 is needed. In one embodiment, the prosthetic femoral neck 200 is in a fixed position, and the head tool 140 is configured to spin the prosthetic femoral head 100 to engage or disengage the threads. In various embodiments, the head tool 140 can apply 0-5000, 0-4000, 0-3000, 0-2000, 0-1000, 0-500, 0-100, 10-80, 20-70, 30-60, 33, 45, and/or 55 ft-lb of torque to the neck thread 250.

As shown in FIG. 7, in one embodiment, a prosthetic hip system 10 includes a neck implant 200 with a neck thread 250 that is connectable to a stem implant 300 with a stem thread 350. In one embodiment, the threads 350, 250 provide a tightenable, locking interface. In one embodiment, the threads 350, 250 are reversible for disassembly. In one embodiment, the threads 350, 250 operate in conjunction with a tapered surface to attach a neck implant 200 to a stem implant 300. In one embodiment, the tapered surfaces 324, 224 are complementary Morse tapers. In one embodiment, a temperature differential 400 is applied to the threaded prosthetic hip system 10.

In one embodiment, the threaded prosthetic hip system 10 is assembled by inserting the prosthetic femoral neck 200 in to the prosthetic femoral stem 300 and rotating the neck 200 and stem 300 with respect to each other to engage the complementary threads 350, 250. As the threads 350, 250 bring the stem 300 and neck 200 together, complementary tapered surfaces 324, 224 can engage each other. With the threaded interface, hammering is not necessary. With the threaded interface, a precise, repeatable attachment can performed with higher precision. In one embodiment, a tool can be configured to deliver a precise or maximum torque to tighten the threads.

In one embodiment, the threads 250, 350 are positioned at a proximal end or near the proximal end of the a prosthetic femoral neck 200 stem engaging end 225 and the neck bore 322 in the prosthetic femoral stem 300. One advantage of positioning threads at the proximal, or “deep” end of the interface is that fretting or debris resulting from micro-motion of the interface localized to the threads will be trapped or contained within the interface. In other embodiments, the threads 250, 350 can be positioned at any point, proximal, medial, distal, or otherwise along the prosthetic femoral neck 200 stem engaging end 225 and the neck bore 322 in the prosthetic femoral stem 300.

In one embodiment, a modular, threaded prosthetic hip system 10 includes a prosthetic femoral neck 2000 with a stem engaging portion 225 that comprises a taper 224 and a thread 250 for attachable engagement to a modular prosthetic femoral stem 300 to the prosthetic femoral neck 200 implant. In one embodiment, the system 10 includes a combination of propelling threads 350, 250 and locking Morse taper surfaces 324, 224 on an axis parallel to, or same as, the longitudinal axis of a modular prosthetic femoral neck 200. In one embodiment, the threads 350, 250 are configured to lock the femoral neck implant 200 component to the femoral stem implant 300 with an interference fit between the threads 350, 250 and the at least one tapered surface 324, 224. In one embodiment, the proximal neck portion includes the stem engaging end, and/or a stem engaging portion. In one embodiment, the stem engaging portion includes a Morse taper and a thread. In one embodiment, the thread 350, 250 redistributes the loading and point of potential micro-motion between the neck 200 and stem 300, creating one, two, three, four, or more pivot points and localizing potential fretting to an isolated, threaded location at the interface. In one embodiment, fretting and materials released by micro-motion are sealed, trapped, or contained within the interface. In one embodiment, fretting and materials are contained within an interface by a taper, such as a Morse taper surface. In one embodiment, the combination of a thread with the taper surfaces provides one, two, three, four, or more point bending that can prevent or reduce micro-motion and reduce fretting and corrosion of the modular connection. In one embodiment, the interface has a two point bending connection. In one embodiment, the interface has a three point bending connection. In one embodiment, the interface has a four point bending connection. In one embodiment, the interface includes a trunnion taper lock. In one embodiment, a combination of propelling threads and locking Morse taper surfaces on the same (or parallel) axis of the modular femoral neck will resolve inaccuracies of manual impaction of the stem onto the neck at the trunnion interface; resulting in consistent reduction of fretting and corrosion.

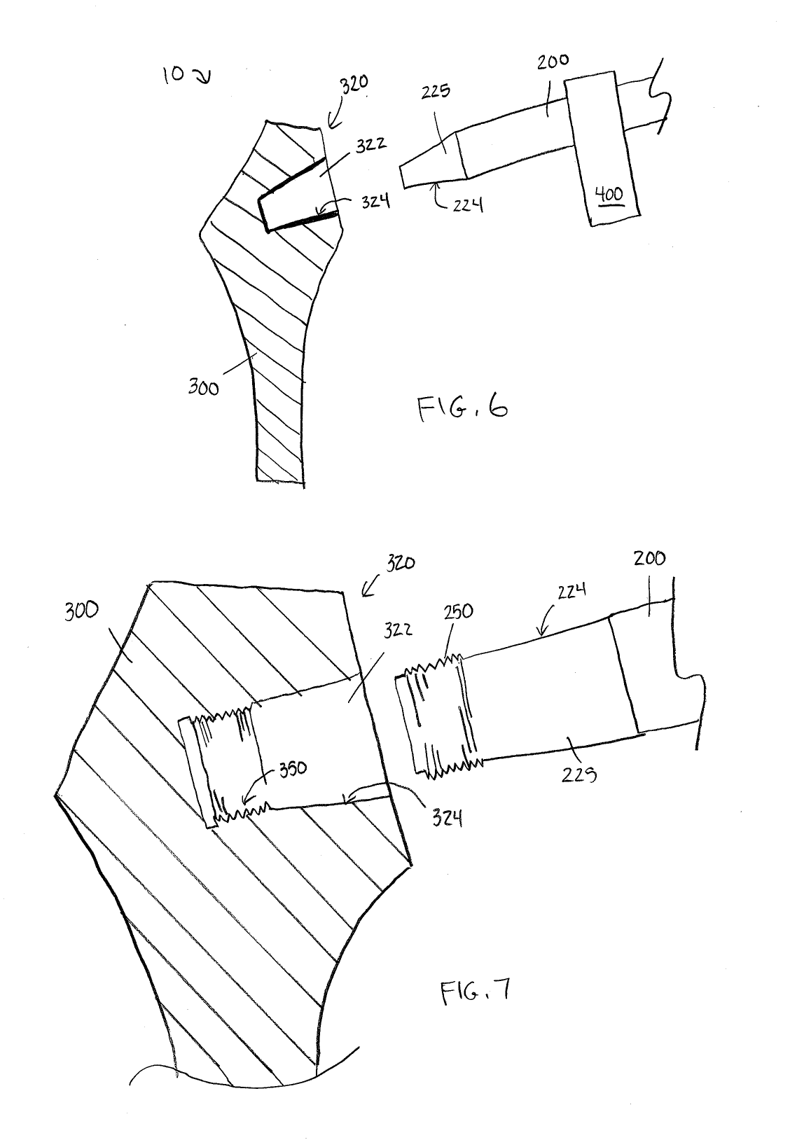

As shown in FIG. 8, in one embodiment, the prosthetic femoral neck 200 includes a neck tool engaging portion 230 configured for a neck tool 240 for implantation, actuation, assembly, rotation, threading, and/or removing the prosthetic femoral neck 200. In various embodiments, the neck tool engaging portion 230 is a slot, keyed interface, hexagonal, or other interface for rotating the prosthetic femoral neck 200 to engage the neck thread 250 with the stem thread 350.

In various embodiments, the neck tool engaging portion 230 can be attached at any point along the prosthetic femoral neck 200, and the neck tool engaging portion 230 includes features for rotatable engagement. In various embodiments, the neck tool 230 can apply 0-5000, 0-4000, 0-3000, 0-2000, 0-1000, 0-500, 0-100, 10-80, 20-70, 30-60, 33, 45, and/or 55 ft-lb of torque to the neck thread 250.

In one embodiment, a stem tool 340 includes one or more pins, keys, or other interface to hold the prosthetic femoral stem 300 in position while a threaded prosthetic femoral neck 200 is threaded to the stem 300.

In one embodiment, as shown at FIG. 9, prosthetic femoral neck is attachable to both a prosthetic femoral head and a prosthetic femoral stem with respective threads and tapered surface interfaces.

Current hip arthroplasty implants, such as dual-taper femoral stems, contain multiple modular components that can be subject to failure due to fretting, corrosion, micromotion at the Morse Taper connection, and the risk of adverse local tissue reaction, pseudo-tumors and even a broken trunnion. These limitations may render these implants potentially harmful to the patient with a number of serious complications.

As such, there is a need in the industry for a durable monoblock apparatus for use as a hip arthroplasty component that reduces the likelihood for fretting and corrosion.

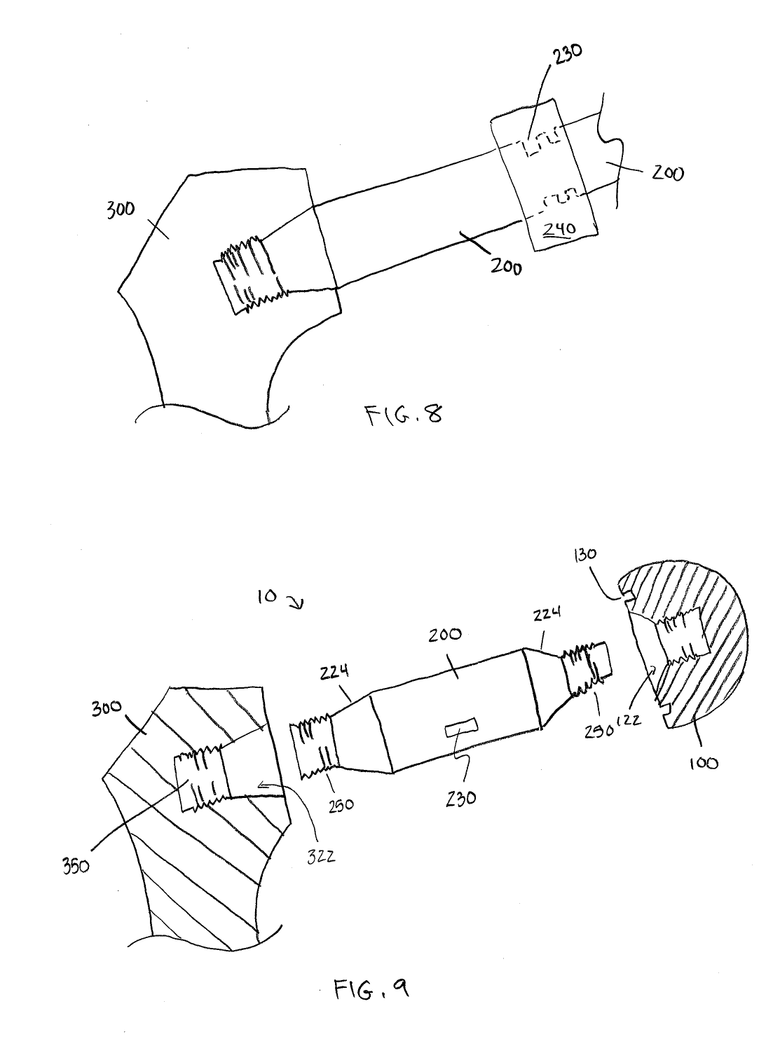

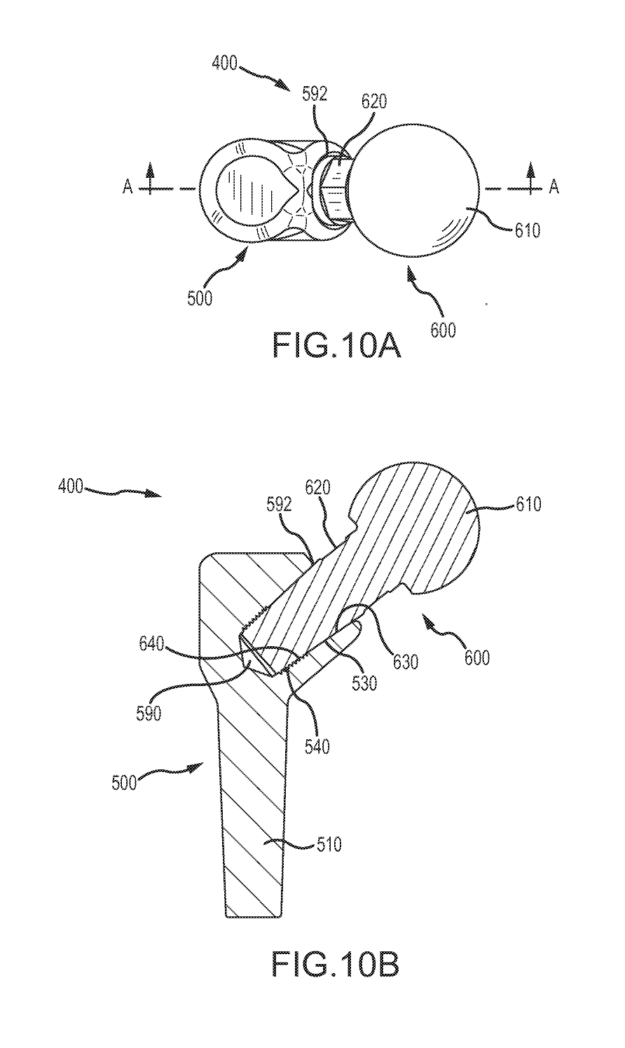

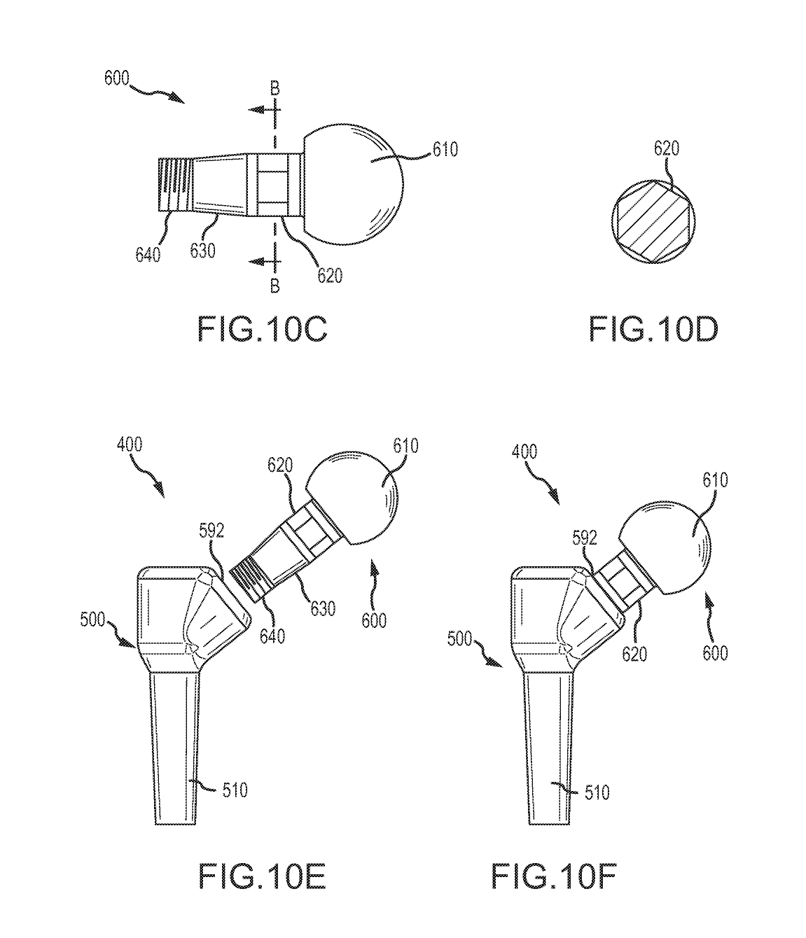

Referring to FIGS. 10A-F, various embodiments of a prosthetic hip system 400 include a femoral stem implant 500, a prosthetic femoral head 600, and a proximal securement member 1000. In various embodiments, each component is configured for insertion through one or more minimally-invasive incisions in patient to reduce the damage to tissue and speed the recovery in a hip replacement. In some embodiments, the stem implant 500 can be monolithically formed with an intramedullary rod portion 510. However, in another embodiment, the stem implant 500 may be formed separately from and subsequently coupled to an intramedullary rod 510.

In some embodiments, the stem implant 500 is configured to taper and define ridges to facilitate engagement and fit into the intramedullary canal of the femur. Further, the stem implant 500 can include ridges, a sleeve (not shown), and/or other structures for engaging the femur and promoting osseointegration, rotational registration, engagement, and other advantageous features. In some embodiments, the femoral stem implant component 500 includes a slot (not shown) to provide additional flexibility in the rod portion 510 of the stem implant along the intramedullary canal. In some embodiments, the stem implant 500 includes an orientation marking (not shown) to indicate the relative position of the stem implant 500 with respect to another component, such as a sleeve. In some embodiments, the stem implant 500 includes an interface configured for temporary attachment to an implant insertion and drill guide assembly. In some embodiments, the interface includes one or more features, recesses, locks, keys, or other aspects for aligning or positioning the stem implant 500 in a particular orientation. In some embodiments, the interface is a thread for releasable positioning and deployment or retrieval of the stem implant 500 with respect to the implant insertion and drill guide assembly. In some embodiments, the interface is a cam.

In some embodiments, the stem implant 500 includes a distal bore 590 extending from a distal bore end 592 at least partially into a body of the stem implant 500. In various embodiments, the distal bore 590 is any shaped interface. In some embodiments, the distal bore 590 is round. In various embodiments, the distal bore 590 is configured to interface with one or more portions of a head and neck unit 600. The distal bore 590 is configured to receive at least a portion of the head and neck unit 600. The distal bore 590 can include one or more registration structures to rotationally secure the head and neck unit 600. The registration structures can include one or more protrusions and/or recesses extending along an outer surface of the head and neck unit 600 and/or the distal bore 590. In some embodiments, the distal bore 590 includes one or more threads (not shown). In some embodiments, the distal bore 590 includes one, two, or more tapered surfaces 530. In some embodiments, the tapered surface 530 is a Morse taper.

In various embodiments, the one or more tapered surfaces 530 are configured to seal respective interfaces between system parts to prevent the escape of debris or flaking from components that may rub against each other in use. Such debris may remain sealed within the bore 590. In various embodiments, the one or more tapered surfaces 530 are configured to provide an adjustable interface to account for differences in tolerances in dimensions between parts or components.

The head and neck unit 600 can extend entirely through the distal bore 590 extend partially or entirely through the distal bore 590.

In some embodiments, the head and neck unit 600 includes a prosthetic femoral head 610, a head and neck unit engagement portion 620, a tapered distal bore engaging portion 630, and a threaded distal bore engaging portion 640. In some embodiments, the head and neck unit engagement portion 620 is configured for a tool to engage the head and neck unit 600 for implantation or removal. In some embodiments, the prosthetic femoral head 610 is integrally formed with all other portions of the head and neck unit 600. For example, the prosthetic femoral head 610 can be integrally formed with the head and neck unit engagement portion 620, the tapered distal bore engaging portion 630, and/or the threaded distal bore engaging portion 640. Where components of the head and neck unit 600 are of a monoblock of material. Interfaces, such as threading and taper surfaces, between components of the head and neck unit 600 can be eliminated. As used herein, a monoblock is a single, integrated object, wherein the components of the object are integrally formed or fixedly attached, such that components of the monoblock are unified in movement and orientation. As compared to modular components requiring an interface between a prosthetic femoral head and a prosthetic femoral neck, removal of such component interfaces minimizes or eliminates the likelihood of fretting and corrosion of the apparatus during use by the patient.

As shown in FIG. 10B, the head and neck unit 600 includes a tapered distal bore engaging portion 630 at a proximal region of the head and neck unit 600. In various embodiments, the tapered distal bore engaging portion 630 can be tapered. In some embodiments, the taper of the tapered distal bore engaging portion 630 can be a Morse taper. In various embodiments, the taper of the tapered distal bore engaging portion 630 can be in the range of 0-10 degrees, 1-9 degrees, 2-8 degrees, 4-7 degrees, 5-6 degrees. Further, a distal section of the distal bore 590 near the distal bore end 592 can also include a corresponding tapered surface 530 for engagement with the tapered distal bore engaging portion 630 of the head and neck unit 600. In various embodiments, one, two, or more tapers of the tapered distal bore engaging portion 630 can extend along between about 0.1-3, 0.5-2, 1-1.5 cm and/or less than or equal to about 3 cm of the head and neck unit 600. In various embodiments, one, two, or more tapered surfaces 530 of the tapered distal bore engaging portion 630 can extend along about 2 cm of the head and neck unit 600. In various embodiments, the diameter of the tapered distal bore engaging portion 630 can be between at least about 10 mm and/or less than or equal to about 17 mm. In some embodiments, the diameter of the tapered distal bore engaging portion 630 can be between at least about 11 mm and/or less than or equal to about 15 mm. Further, the diameter of the distal section of the distal bore 590 can be between at least about 10 mm and/or less than or equal to about 17 mm. In some embodiments, the diameter of the distal section of the distal bore 590 can be between at least about 11 mm and/or less than or equal to about 15 mm. The diameter of the distal section of the distal bore 590 and the diameter of the tapered distal bore engaging portion 630 can increase gradually as the bore extends toward the prosthetic femoral head 610 and/or the distal bore end 592 to accommodate the Morse taper. For example, the distal bore 590 can have a cross-sectional dimension at the distal bore end 592 that is greater than a cross-sectional dimension at a location within the distal bore 590 that is proximal to the distal bore end 592. In some embodiments, the diameters of the tapered distal bore engaging portion 630 and the distal section of the distal bore 590 can define a generally identical or complementary taper and geometry. For example, the tapering of the tapered distal bore engaging portion 630 and the distal section of the distal bore 590 can be linear or define an arcuate (either increasingly or decreasingly smaller diameter) tapered surface 530.

As shown in FIG. 10B, in some embodiments, the head and neck unit 600 includes a threaded distal bore engaging portion 640 that is connectable to a stem implant 500 with a stem thread 540. In some embodiments, the threads 540, 640 provide a tightenable, locking interface. In some embodiments, the threads 540, 640 are reversible for disassembly. In some embodiments, the threads 540, 640 operate in conjunction with the tapered distal bore engaging portion 630 and the tapered surface 530 to attach the head and neck unit 600 to the stem implant 500. In some embodiments, the tapered surfaces 530, 630 are complementary Morse tapers.

In some embodiments, the threads 640, 540 are positioned at a proximal end or near the proximal end of the head and neck unit 600 and the distal bore 590 in the stem implant 500. One advantage of positioning threads at the proximal, or “deep” end of the interface is that fretting or debris resulting from micro-motion of the interface localized to the threads will be trapped or contained within the interface. In other embodiments, the threads 640, 540 can be positioned at any point, proximal, medial, distal, or otherwise along the head and neck unit 600 and the distal bore 590 in the stem implant 500. For example, the threaded distal bore engaging portion 640 can be located axially between the tapered distal bore engaging portion 630 and the prosthetic femoral head 610. As shown in FIG. 10B-C, the tapered distal bore engaging portion 630 can be located axially between the threaded distal bore engaging portion 640 and the prosthetic femoral head 610.

In some embodiments, the threads 540, 640 and tapers 530, 630 are on an axis parallel to, or same as, the longitudinal axis of the head and neck unit 600. In some embodiments, the threads 540, 640 are configured to lock the head and neck unit 600 to the stem implant 500 with an interference fit between the threads 540, 640 and the tapers 530, 630. In some embodiments, the thread 540, 640 redistributes the loading and point of potential micro-motion between the head and neck unit 600 and the stem implant 500, creating one, two, three, four, or more pivot points and localizing potential fretting to an isolated, threaded location at the interface. In some embodiments, fretting and materials released by micro-motion are sealed, trapped, or contained within the interface. In some embodiments, fretting and materials are contained within an interface by a taper, such as a Morse taper surface. In some embodiments, the combination of a thread with the taper surfaces provides one, two, three, four, or more point bending that can prevent or reduce micro-motion and reduce fretting and corrosion of the modular connection. In some embodiments, the interface has a two point bending connection. In some embodiments, the interface has a three point bending connection. In some embodiments, the interface has a four point bending connection. In some embodiments, the interface includes a trunnion taper lock. In some embodiments, a combination of propelling threads and locking Morse taper surfaces on the same (or parallel) axis of the modular femoral neck will resolve inaccuracies of manual impaction of the stem onto the neck at the trunnion interface; resulting in consistent reduction of fretting and corrosion.

In some embodiments, the components of the prosthetic hip system 400 are provided to a patient during a surgical procedure. The stem implant 500 engages a femur of the patient. As shown in FIGS. 10E-F, the head and neck unit 600 is inserted into the distal bore 590. The head and neck unit 600 is threadably engaged with the distal bore 590 by engaging the neck unit engagement portion 620 with a tool. Relative rotation of the head and neck unit 600 and the stem implant 800 is achieved to create relative axial movement of the same. In some embodiments, the threaded distal bore engaging portion 640 is threadably engaged with the threads 540 of the distal bore 590 until the taper of the tapered distal bore engaging portion 630 and the taper 530 of the distal bore 590 match against each other. The head 610 of the head and neck unit 600 is provided to an acetabulum region of the patient.

Optionally, the head and neck unit 600 can be exposed to a temperature differential to cool the head and neck unit 600 to reduce at least one dimension of the head and neck unit 600 through thermal contraction. In some embodiments, the head and neck unit 600 the neck is cooled in a cooling medium, such as liquid nitrogen, prior to inserting the head and neck unit 600 in the distal bore 590 of the femoral stem implant component 500. In some embodiments, the femoral stem implant component 500 can receive the head and neck unit 600 in a cooled, contracted state, at which time the head and neck unit 600 will be shrunk to a reduced dimensional geometry. The head and neck unit 600 can then be installed into the distal bore 590 until an interference fit is obtained between the head and neck unit 600 and the distal bore 590. The interference fit can be achieved due to interaction of corresponding engagement structures, such as threads, Morse tapers, protrusions, recesses, and other such geometries and corresponding features. In such embodiments, the engagement between the neck and the support sleeve can provide superior strength and permanence. In some embodiments, a temperature differential can be used in conjunction with one, two, or more Morse tapers that are configured to interact between components to cause an interference fit and/or cold welding to achieve exceptional engagement as the cooled component(s) enlarge when exposed to the body temperature, warming and expanding components.

In various embodiments, one or more threads 540, 640 are sized with a pitch and dimensions configured to be rotatably threadable with respect to a corresponding thread at an ambient, body, and/or cooled temperature. In some embodiments, one or more threads 540, 640 are rotatable when cooled to a threshold temperature under a temperature differential, and lock in place with an interference fit or cold welding when heated to ambient or body temperature. In various embodiments, monitoring of component temperature and/or dimensions may be involved in a hip arthroplasty procedure.

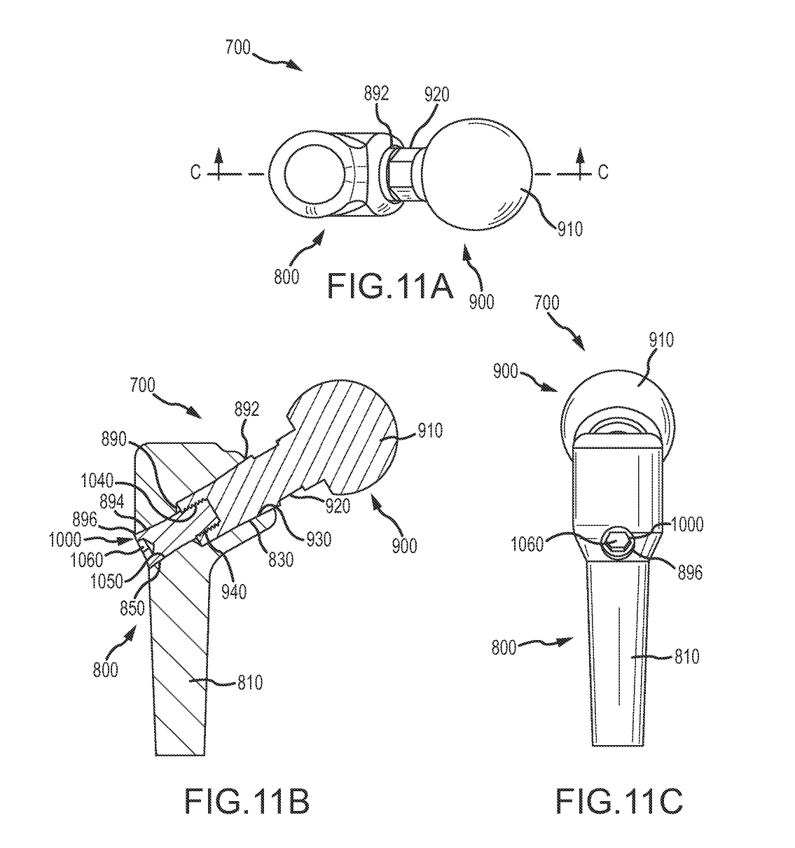

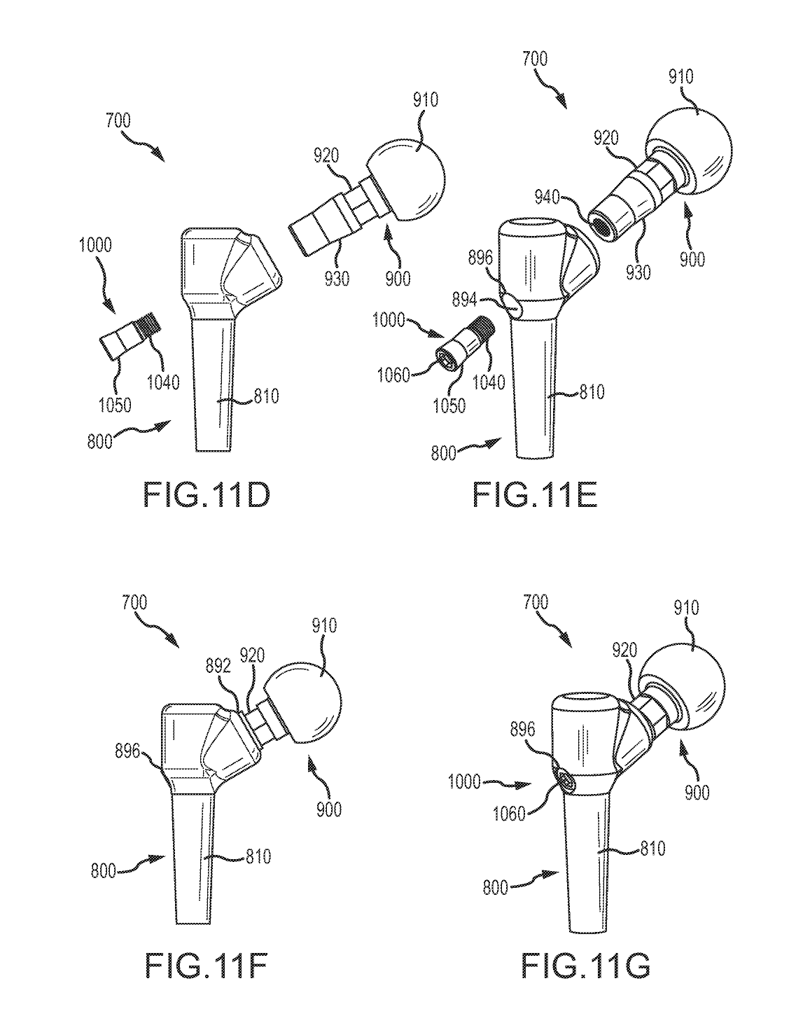

Referring to FIGS. 11A-G, various embodiments of a prosthetic hip system 700 include a femoral stem implant 800, a prosthetic femoral head 900, and a proximal securement member 1000. In various embodiments, each component is configured for insertion through one or more minimally-invasive incisions in patient to reduce the damage to tissue and speed the recovery in a hip replacement. In some embodiments, the stem implant 800 can be monolithically formed with an intramedullary rod portion 810. However, in another embodiment, the stem implant 800 may be formed separately from and subsequently coupled to an intramedullary rod 810.

In some embodiments, the stem implant 800 is configured to taper and define ridges to facilitate engagement and fit into the intramedullary canal of the femur. Further, the stem implant 800 can include ridges, a sleeve (not shown), and/or other structures for engaging the femur and promoting osseointegration, rotational registration, engagement, and other advantageous features. In some embodiments, the femoral stem implant component 800 includes a slot (not shown) to provide additional flexibility in the rod portion 810 of the stem implant along the intramedullary canal. In some embodiments, the stem implant 800 includes an orientation marking (not shown) to indicate the relative position of the stem implant 800 with respect to another component, such as a sleeve. In some embodiments, the stem implant 800 includes an interface configured for temporary attachment to an implant insertion and drill guide assembly. In some embodiments, the interface includes one or more features, recesses, locks, keys, or other aspects for aligning or positioning the stem implant 800 in a particular orientation. In some embodiments, the interface is a thread for releasable positioning and deployment or retrieval of the stem implant 800 with respect to the implant insertion and drill guide assembly. In some embodiments, the interface is a cam.

In some embodiments, the stem implant 800 includes a distal bore 890 extending from a distal bore end 892 at least partially into a body of the stem implant 800. In various embodiments, the distal bore 890 is any shaped interface. In some embodiments, the distal bore 890 is round. In various embodiments, the distal bore 890 is configured to interface with one or more portions of a head and neck unit 900. The distal bore 890 is configured to receive at least a portion of the head and neck unit 900. The distal bore 890 can include one or more registration structures to rotationally secure the head and neck unit 900. The registration structures can include one or more protrusions and/or recesses extending along an outer surface of the head and neck unit 900 and/or the distal bore 890. In some embodiments, the distal bore 890 includes one or more threads (not shown). In some embodiments, the distal bore 890 includes one, two, or more tapered surfaces 830. In some embodiments, the tapered surface 830 is a Morse taper.

In some embodiments, the stem implant 800 includes a proximal bore 894 extending from a proximal bore end 896 at least partially into a body of the stem implant 800. In various embodiments, the proximal bore 894 is any shaped interface. In some embodiments, the proximal bore 894 is round. In various embodiments, the proximal bore 894 is configured to interface with one or more portions of a proximal securing member 1000. The proximal bore 894 is configured to receive at least a portion of the proximal securing member 1000. The proximal bore 894 can include one or more registration structures to rotationally secure the proximal securing member 1000. The registration structures can include one or more protrusions and/or recesses extending along an outer surface of the proximal securing member 1000 and/or the proximal bore 894. In some embodiments, the proximal bore 894 includes one or more threads (not shown). In some embodiments, the proximal bore 894 includes one, two, or more tapered surfaces 850. In some embodiments, the tapered surface 850 is a Morse taper.

In various embodiments, the tapered surfaces 830, 850 are configured to seal respective interfaces between system parts to prevent the escape of debris or flaking from components that may rub against each other in use. Such debris may remain sealed within the bores 890, 894 between the tapered surfaces 830, 850. In various embodiments, the tapered surfaces 830, 850 are configured to provide an adjustable interface to account for differences in tolerances in dimensions between parts or components.

An axis of the proximal bore 894 may be parallel to or coaxial with an axis of the distal bore 890. The distal bore 890 and the proximal bore 894 may be overlapping, such that a passageway is formed extending through both of the distal bore 890 and the proximal bore 894. The proximal securing member 1000 can extend entirely through the proximal bore 894 and into the distal bore 890. Alternatively, the proximal securing member 1000 can extend only partially through the proximal bore 894 (not shown). The head and neck unit 900 can extend entirely through the distal bore 890 and into the proximal bore 894 (not shown). Alternatively, the head and neck unit 900 can extend only partially through the distal bore 890.

In some embodiments, the head and neck unit 900 includes a prosthetic femoral head 910, a head and neck unit engagement portion 920, and a distal bore engaging portion 930. In some embodiments, the head and neck unit engagement portion 920 is configured for a tool to engage the head and neck unit 900 for implantation or removal. In some embodiments, the prosthetic femoral head 910 is integrally formed with all other portions of the head and neck unit 900. For example, the prosthetic femoral head 910 can be integrally formed with the head and neck unit engagement portion 920 and/or the distal bore engaging portion 930. Where components of the head and neck unit 900 are of a monoblock of material. Interfaces, such as threading and taper surfaces, between components of the head and neck unit 900 can be eliminated. As compared to modular components requiring an interface between a prosthetic femoral head and a prosthetic femoral neck, removal of such component interfaces minimizes or eliminates the likelihood of fretting and corrosion of the apparatus during use by the patient.

As shown in FIG. 11B, the head and neck unit 900 includes a distal bore engaging portion 930 at a proximal region of the head and neck unit 900. In various embodiments, the distal bore engaging portion 930 can be tapered. In some embodiments, the taper of the distal bore engaging portion 930 can be a Morse taper. In various embodiments, the taper of the distal bore engaging portion 930 can be in the range of 0-10 degrees, 1-9 degrees, 2-8 degrees, 4-7 degrees, 5-6 degrees. Further, a distal section of the distal bore 890 near the distal bore end 892 can also include a corresponding tapered surface 830 for engagement with the distal bore engaging portion 930 of the head and neck unit 900. In various embodiments, one, two, or more tapers of the distal bore engaging portion 930 can extend along between about 0.1-3, 0.5-2, 1-1.5 cm and/or less than or equal to about 3 cm of the head and neck unit 900. In various embodiments, one, two, or more tapered surfaces 830 of the distal bore engaging portion 930 can extend along about 2 cm of the head and neck unit 900. In various embodiments, the diameter of the distal bore engaging portion 930 can be between at least about 10 mm and/or less than or equal to about 17 mm. In some embodiments, the diameter of the distal bore engaging portion 930 can be between at least about 11 mm and/or less than or equal to about 15 mm. Further, the diameter of the distal section of the distal bore 890 can be between at least about 10 mm and/or less than or equal to about 17 mm. In some embodiments, the diameter of the distal section of the distal bore 890 can be between at least about 11 mm and/or less than or equal to about 15 mm. The diameter of the distal section of the distal bore 890 and the diameter of the distal bore engaging portion 930 can increase gradually as the bore extends toward the prosthetic femoral head 910 and/or the distal bore end 892 to accommodate the Morse taper. For example, the distal bore 890 can have a cross-sectional dimension at the distal bore end 892 that is greater than a cross-sectional dimension at a location within the distal bore 890 that is proximal to the distal bore end 892. In some embodiments, the diameters of the distal bore engaging portion 930 and the distal section of the distal bore 890 can define a generally identical or complementary taper and geometry. For example, the tapering of the distal bore engaging portion 930 and the distal section of the distal bore 890 can be linear or define an arcuate (either increasingly or decreasingly smaller diameter) tapered surface 830.

In some embodiments, a proximal end of the head and neck unit 900 includes a structure for engaging a distal end of the proximal securing member 1000. The proximal securing member 1000 can include a corresponding engagement structure that facilitates engagement with the engagement structure of the head and neck unit 900. In some embodiments, the head and neck unit 900 has a thread 940. The thread 940 may be on an internal surface of the head and neck unit 900. Alternatively, the thread 940 may be on an external surface of the head and neck unit 900 (not shown). In some embodiments, the proximal securing member 1000 has a thread 1040. The thread 1040 may be on an external surface of the proximal securing member 1000. Alternatively, the thread 1040 may be on an internal surface of the proximal securing member 1000 (not shown). In some embodiments, the engagement structures include corresponding threads 940, 1040. The threads 940, 1040 can allow the proximal securing member 1000 to be rotated onto the head and neck unit 900 with some adjustability. In some embodiments, the proximal securing member 1000 includes a proximal securing member engagement structure 1060 at its proximal end that facilitates engagement with a tool to install, remove, tighten, and/or loosen the proximal securing member 1000. In some embodiments, the proximal securing member engagement structure 1060 includes features for rotatable engagement. In various embodiments, the proximal securing member engagement structure 1060 can apply 0-100, 10-80, 20-70, 30-60, 33, 45, and/or 55 ft-lb of torque to the proximal securing member 1000.

As shown in FIG. 11B, the proximal securing member 1000 includes a proximal bore engaging portion 1050. In various embodiments, the proximal bore engaging portion 1050 can be tapered. In some embodiments, the taper of proximal bore engaging portion 1050 can be a Morse taper. In various embodiments, the taper of the proximal bore engaging portion 1050 can be in the range of 0-10 degrees, 1-9 degrees, 2-8 degrees, 4-7 degrees, 5-6 degrees. Further, a distal section of the proximal bore 894 near the distal bore end 892 can also include a corresponding tapered surface 1050 for engagement with the proximal bore engaging portion 1050 of the proximal securing member 1000. In various embodiments, one, two, or more tapers of the proximal bore engaging portion 1050 can extend along between about 0.1-3, 0.5-2, 1-1.5 cm and/or less than or equal to about 3 cm of the proximal securing member 1000. In various embodiments, one, two, or more tapered surfaces 1050 of the proximal bore engaging portion 1050 can extend along about 2 cm of the proximal securing member 1000. In various embodiments, the diameter of the proximal bore engaging portion 1050 can be between at least about 10 mm and/or less than or equal to about 17 mm. In some embodiments, the diameter of the proximal bore engaging portion 1050 can be between at least about 11 mm and/or less than or equal to about 15 mm. Further, the diameter of the proximal section of the proximal bore 894 can be between at least about 10 mm and/or less than or equal to about 17 mm. In some embodiments, the diameter of the proximal section of the proximal bore 894 can be between at least about 11 min and/or less than or equal to about 15 mm. The diameter of the proximal section of the proximal bore 894 and the diameter of the proximal bore engaging portion 1050 can increase gradually as the bore extends toward the proximal bore end 896 to accommodate the Morse taper. For example, the proximal bore 894 can have a cross-sectional dimension at the proximal bore end 896 that is greater than a cross-sectional dimension at a location within the proximal bore 894 that is distal to the proximal bore end 896. In some embodiments, the diameters of the proximal bore engaging portion 1050 and the distal section of the proximal bore 894 can define a generally identical or complementary taper and geometry. For example, the tapering of the proximal bore engaging portion 1050 and the distal section of the proximal bore 894 can be linear or define an arcuate (either increasingly or decreasingly smaller diameter) tapered surface 1050.

In some embodiments, at least a portion (e.g., the distal bore end 892) of the distal bore 890 can define a larger diameter than at least a portion (e.g., the proximal bore end 896) of the proximal bore 894.

In some embodiments, the components of the prosthetic hip system 700 are provided to a patient during a surgical procedure. The stem implant 800 engages a femur of the patient. As shown in FIGS. 11D-G, the head and neck unit 900 is inserted into the distal bore 890, and the proximal securing member 1000 is inserted into the proximal bore 894. The head and neck unit 900 is threadably engaged with the proximal securing member 1000 by engaging the neck unit engagement portion 920 with a first tool and engaging the proximal securing member engagement structure 1060 with a second tool. Relative rotation of the head and neck unit 900 and the proximal securing member 1000 is achieved to create relative axial movement of the same. In some embodiments, the head and neck unit 900 is threadably engaged with the proximal securing member 1000 until one or more tapers of the distal bore engaging portion 930 and the distal bore 890 match against each other. In some embodiments, the head and neck unit 900 is threadably engaged with the proximal securing member 1000 until one or more tapers of the proximal bore engaging portion 1050 and the proximal bore 894 match against each other. The head 910 of the head and neck unit 900 is provided to an acetabulum region of the patient.

In some embodiments, the engagement and relative adjustment of the head and neck unit 900 and the proximal securing member 1000 increases or decreases a degree of engagement between the proximal bore engaging portion 1050 of the proximal securing member 1000 and the tapered surface 850 of the proximal bore 894. In some embodiments, the engagement and relative adjustment of the head and neck unit 900 and the proximal securing member 1000 increases or decreases a degree of engagement between the distal bore engaging portion 930 of the head and neck unit 900 and the tapered surface 830 of the distal bore 890. For example, as the head and neck unit 900 and the proximal securing member 1000 are brought into an engaged condition, the head and neck unit 900 and the proximal securing member 1000 are moved toward each other. Accordingly, the proximal bore engaging portion 1050 of the proximal securing member 1000 is brought into further engagement with the tapered surface 850 of the proximal bore 894, and the distal bore engaging portion 930 of the head and neck unit 900 is brought into further engagement with the tapered surface 830 of the distal bore 890. A force applied by the proximal bore engaging portion 1050 on the tapered surface 850 can be equal to a force applied by the distal bore engaging portion 930 on the tapered surface 830.

By further example, as the head and neck unit 900 and the proximal securing member 1000 are released from an engaged condition, the head and neck unit 900 and the proximal securing member 1000 are moved away from each other. Accordingly, the proximal bore engaging portion 1050 of the proximal securing member 1000 is released from engagement with the tapered surface 850 of the proximal bore 894, and/or the distal bore engaging portion 930 of the head and neck unit 900 is released from engagement with the tapered surface 830 of the distal bore 890.

Optionally, the head and neck unit 900 can be exposed to a temperature differential to cool the head and neck unit 900 to reduce at least one dimension of the head and neck unit 900 through thermal contraction. In some embodiments, the head and neck unit 900 the neck is cooled in a cooling medium, such as liquid nitrogen, prior to inserting the head and neck unit 900 in the distal bore 890 of the femoral stem implant component 800. In some embodiments, the femoral stem implant component 800 can receive the head and neck unit 900 in a cooled, contracted state, at which time the head and neck unit 900 will be shrunk to a reduced dimensional geometry. The head and neck unit 900 can then be installed into the distal bore 890 until an interference fit is obtained between the head and neck unit 900 and the distal bore 890. The interference fit can be achieved due to interaction of corresponding engagement structures, such as threads, Morse tapers, protrusions, recesses, and other such geometries and corresponding features. In such embodiments, the engagement between the neck and the support sleeve can provide superior strength and permanence. In some embodiments, a temperature differential can be used in conjunction with one, two, or more Morse tapers that are configured to interact between components to cause an interference fit and/or cold welding to achieve exceptional engagement as the cooled component(s) enlarge when exposed to the body temperature, warming and expanding components.

In some embodiments, the method includes installing the proximal securing member 1000 of the head and neck unit 900 after the proximal securing member 1000 has been cooled in a cooling medium. The proximal securing member 1000 can be threadably engaged with the head and neck unit 900 during installation. In some embodiments, the head and neck unit 900 and proximal securing member 1000 are installed in quick successive order in order to ensure that both are at a cool temperature when initially engaged with each other. Thus, as the head and neck unit 900 and proximal securing member 1000 warm from the cooled temperature, the engagement sections (e.g. threads, tapers, etc.) can expand against each other to create an interference fit that secures the head and neck unit 900 and proximal securing member 1000 together with a superior, strong bond.

In various embodiments, one or more threads 940, 1040 are sized with a pitch and dimensions configured to be rotatably threadable with respect to a corresponding thread at an ambient, body, and/or cooled temperature. In some embodiments, one or more threads 940, 1040 are rotatable when cooled to a threshold temperature under a temperature differential, and lock in place with an interference fit or cold welding when heated to ambient or body temperature. In various embodiments, monitoring of component temperature and/or dimensions may be involved in a hip arthroplasty procedure.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A prosthetic hip system for hip arthroplasty, comprising:
a stem implant comprising an intramedullary rod and a distal bore extending into the stem implant from a distal bore end, the distal bore defining a tapered surface and a thread;
a monoblock head and neck unit comprising a prosthetic femoral head, a tapered distal bore engaging portion configured to engage the tapered surface, and a threaded distal bore engaging portion configured to engage the thread.

2. The prosthetic hip system of claim 1, wherein the intramedullary rod is configured to engage a femur of a patient, and the distal bore end is configured to face an acetabular region of the patient while the intramedullary rod engages the femur.

3. The prosthetic hip system of claim 1, wherein the monoblock head and neck unit further comprises a neck unit engagement portion configured to be engaged by a tool.

4. The prosthetic hip system of claim 1, wherein adjustment of the threaded distal bore engaging portion within the thread adjusts a degree of engagement between the tapered distal bore engaging portion and the tapered surface.

5. The prosthetic hip system of claim 1, wherein the tapered distal bore engaging portion is axially between the threaded distal bore engaging portion and the prosthetic femoral head.

6. The prosthetic hip system of claim 1, wherein the tapered surface is axially between the thread and the distal bore end.

7. The prosthetic hip system of claim 1, wherein an engagement between the tapered distal bore engaging portion and the tapered surface is configured to seal an interior region of the stem implant.

8. The prosthetic hip system of claim 1, wherein a first cross-sectional dimension at the distal bore end is greater than a second cross-sectional dimension at a location within the distal bore and proximal to the distal bore end.

9. A method of implanting a prosthetic hip system for hip arthroplasty, comprising:
engaging a femur of a patient with an intramedullary rod of a stem implant;
inserting a portion of a monoblock head and neck unit into a distal bore of the stem implant through a distal bore end, the monoblock head and neck unit comprising a prosthetic femoral head, a tapered distal bore engaging portion, and a threaded distal bore engaging portion;
engaging the threaded distal bore engaging portion with a thread of the distal bore; and
adjusting the threaded distal bore engaging portion relative to the thread, such that the tapered distal bore engaging portion engages a tapered surface of the distal bore.

10. The method of claim 9, further comprising aligning the prosthetic femoral head with an acetabular region of the patient, such that the distal bore end faces the acetabular region.

11. The method of claim 9, wherein a first cross-sectional dimension at the distal bore end is greater than a second cross-sectional dimension at a location within the distal bore and proximal to the distal bore end.

12. A prosthetic hip system for hip arthroplasty, comprising:
a stem implant comprising an intramedullary rod, a distal bore extending into the stem implant from a distal bore end, the distal bore defining a distal tapered surface, and a proximal bore extending into the stem implant from a proximal bore end, the proximal bore defining a proximal tapered surface and intersecting the distal bore;
a monoblock head and neck unit comprising a prosthetic femoral head, a tapered distal bore engaging portion configured to engage the distal tapered surface, and a first thread; and
a proximal securing member comprising a tapered proximal bore engaging portion configured to engage the proximal tapered surface, and a second thread configured to engage the first thread.

13. The prosthetic hip system of claim 12, wherein the intramedullary rod is configured to engage a femur of a patient, and the distal bore end is configured to face an acetabular region of the patient while the intramedullary rod engages the femur.

14. The prosthetic hip system of claim 12, wherein the monoblock head and neck unit further comprises a neck unit engagement portion configured to be engaged by a first tool, and wherein the proximal securing member comprises a securing member engagement portion configured to be engaged by a second tool.

15. The prosthetic hip system of claim 12, wherein adjustment of the first thread relative to the second thread adjusts a degree of engagement between the tapered distal bore engaging portion and the distal tapered surface and a degree of engagement between the tapered proximal bore engaging portion and the proximal tapered surface.

16. The prosthetic hip system of claim 12, wherein an engagement between the tapered distal bore engaging portion and the distal tapered surface and an engagement between the tapered proximal bore engaging portion and the proximal tapered surface is configured to seal an interior region of the stem implant.

17. The prosthetic hip system of claim 12, wherein a first cross-sectional dimension at the distal bore end is greater than a second cross-sectional dimension at a location within the distal bore and proximal to the distal bore end; and wherein a third cross-sectional dimension at the proximal bore end is greater than a fourth cross-sectional dimension at a location within the proximal bore and distal to the proximal bore end.

18. A method of implanting a prosthetic hip system for hip arthroplasty, comprising:
engaging a femur of a patient with an intramedullary rod of a stem implant;
inserting a portion of a monoblock head and neck unit into a distal bore of the stem implant through a distal bore end, the monoblock head and neck unit comprising a prosthetic femoral head, a tapered distal bore engaging portion, and a first thread;
inserting a portion of a proximal securing member into a proximal bore of the stem implant through a proximal bore end, the proximal securing member comprising a tapered proximal bore engaging portion and a second thread;
engaging the first thread with the second thread; and
adjusting the first thread relative to the second thread, such that the tapered distal bore engaging portion engages a distal tapered surface of the distal bore and such that the tapered proximal bore engaging portion engages a proximal tapered surface of the proximal bore.

19. The method of claim 18, further comprising aligning the prosthetic femoral head with an acetabular region of the patient, such that the distal bore end faces the acetabular region.

20. The method of claim 18, wherein a first cross-sectional dimension at the distal bore end is greater than a second cross-sectional dimension at a location within the distal bore and proximal to the distal bore end; and wherein a third cross-sectional dimension at the proximal bore end is greater than a fourth cross-sectional dimension at a location within the proximal bore and distal to the proximal bore end.

21. The method of claim 18, wherein the proximal securing member extends at least partially into the distal bore and the engaging the first thread with the second thread occurs in the distal bore.
